(19) United States
(12) Patent Application Publication
(10) Pub. No.: US 2026/0097065 A1
(43) Pub. Date: Apr. 9, 2026

(54) HYDROXYPROPYL BETA-CYCLODEXTRIN COMPOSITIONS AND METHODS

(71) Applicant: Mandos LLC, Thousand Oaks, CA (US)

(72) Inventors: Bernardus Nicolaas Machielse, North Potomac, MD (US); Allan Darling, North Potomac, MD (US)

(21) Appl. No.: 19/406,190

(22) Filed: Dec. 2, 2025

Related U.S. Application Data
(63) Continuation of application No. 19/312,906, filed on Aug. 28, 2025.
Application No. 19/312,906 is a continuation of application No. 19/219,596, filed on May 27, 2025, now Pat. No. 12,478,635.
Application No. 19/219,596 is a continuation of application No. 19/028,631, filed on Jan. 17, 2025, now Pat. No. 12,414,964.
Application No. 19/028,631 is a continuation of application No. 18/533,373, filed on Dec. 8, 2023.
Application No. 18/533,373 is a continuation of application No. 17/970,080, filed on Oct. 20, 2022, now Pat. No. 11,938,144.
Application No. 17/970,080 is a continuation of application No. 17/745,464, filed on May 16, 2022, now Pat. No. 11,590,159.
Application No. 17/745,464 is a continuation of application No. 17/036,148, filed on Sep. 29, 2020, now Pat. No. 11,406,657.
Application No. 17/036,148 is a continuation of application No. 16/430,664, filed on Jun. 4, 2019, now Pat. No. 10,933,083.
Application No. 16/430,664 is a continuation of application No. 16/372,899, filed on Apr. 2, 2019, now Pat. No. 10,709,730.
Application No. 16/372,899 is a continuation of application No. 16/134,028, filed on Sep. 18, 2018, now Pat. No. 10,300,086.
Application No. 16/134,028 is a continuation of application No. 15/499,831, filed on Apr. 27, 2017, now Pat. No. 10,258,641.
Application No. 15/499,831 is a continuation of application No. 15/288,876, filed on Oct. 7, 2016, now Pat. No. 9,675,634.
Application No. 15/288,876 is a continuation of application No. 15/178,153, filed on Jun. 9, 2016, now abandoned.
(60) Provisional application No. 62/173,889, filed on Jun. 10, 2015.
(60) Provisional application No. 62/175,075, filed on Jun. 12, 2015.
(60) Provisional application No. 62/189,114, filed on Jul. 6, 2015.
(60) Provisional application No. 62/245,974, filed on Oct. 23, 2015.
(60) Provisional application No. 62/249,876, filed on Nov. 2, 2015.
(60) Provisional application No. 62/263,599, filed on Dec. 4, 2015.
(60) Provisional application No. 62/276,728, filed on Jan. 8, 2016.
(60) Provisional application No. 62/308,736, filed on Mar. 15, 2016.
(60) Provisional application No. 62/314,765, filed on Mar. 29, 2016.
(60) Provisional application No. 62/331,385, filed on May 3, 2016.
(60) Provisional application No. 62/345,721, filed on Jun. 3, 2016.

(51) Int. Cl. A61K 31/724; A61K 9/00; A61K 9/08

(52) U.S. Cl. CPC A61K 31/724; A61K 9/0019; A61K 9/0085; A61K 9/08

(57) ABSTRACT

The present invention provides a maackiain-hydroxypropyl-β-cyclodextrin inclusion compound and a preparation method thereof, and belongs to the field of pharmaceuticals. In the present invention, water and hydroxypropyl-β-cyclodextrin are sequentially added to a maackiain solution, and stirred in darkness to obtain a maackiain-hydroxypropyl-β-cyclodextrin inclusion compound. The solvent of the maackiain solution is an organic solvent. In the present invention, hydroxypropyl-β-cyclodextrin is used to include maackiain, and maackiain enters from the small opening end of hydroxypropyl-β-cyclodextrin and penetrates through the cavity of hydroxypropyl-β-cyclodextrin to form the inclusion compound, which can significantly improve the stability and solubility of maackiain.

### FIELD OF THE INVENTION

The present invention relates to the field of pharmaceutical technology, and in particular to a maackiain-hydroxypropyl-β-cyclodextrin inclusion compound and a preparation method thereof.

### BACKGROUND OF THE INVENTION

Maackiain (C₁₆H₁₂O₅, MAA for short) is widely found in leguminous plants and is one of the active ingredients of Sophora flavescens. It has strong anti-allergic and anti-cancer effects, can induce cell apoptosis, and can also be used to treat cholecystitis and arthritis. In addition, it is also used as an insecticide and antitoxin, and is favored by scientists.

However, MAA has poor solubility, is relatively turbid when used as in the form of a medicine liquid in that the liquid has unqualified clarity and poor stability, and is prone to mildew. It is difficult to make it into oral dosage forms. At present, it is mainly made into granules, which limits its application scope.

### SUMMARY OF THE INVENTION

In view of this, an objective of the present invention is to provide a maackiain-hydroxypropyl-β-cyclodextrin inclusion compound and a preparation method thereof. The present invention uses hydroxypropyl-β-cyclodextrin to include maackiain, and after the inclusion compound is formed, the solubility and stability of maackiain are significantly improved.

In order to realize the above-mentioned objective of the invention, the present invention provides the following technical solutions:

A method for preparing a maackiain-hydroxypropyl-β-cyclodextrin inclusion compound, comprising the following steps:
adding water and hydroxypropyl-β-cyclodextrin to a maackiain solution in sequence, and stirring the mixture in darkness to obtain a maackiain-hydroxypropyl-β-cyclodextrin inclusion compound;
the solvent of the maackiain solution is an organic solvent.

Preferably, the organic solvent includes one or more of methanol, ethanol, isopropyl alcohol, acetonitrile and acetone.

Preferably, the molar ratio of maackiain to hydroxypropyl-β-cyclodextrin in the maackiain solution is 1:1 to 1:2.

Preferably, the concentration of the maackiain solution is 2.125-4.25 mg/mL.

Preferably, the volume ratio of the maackiain solution to water is 2-4:8-10.

Preferably, the time of the stirring in darkness is 1 h to 5 h.

Preferably, the stirring in darkness further comprises: subjecting the resulting stirred liquid to filtration, rotary evaporation and drying in sequence.

Preferably, the temperature of the rotary evaporation is 55-70° C.

The present invention provides a maackiain-hydroxypropyl-β-cyclodextrin inclusion compound prepared by the preparation method described in the above solutions.

The present invention provides a method for preparing a maackiain-hydroxypropyl-β-cyclodextrin inclusion compound. According to the present invention, water and hydroxypropyl-β-cyclodextrin are added to a maackiain solution in sequence, stirred in darkness to obtain a maackiain-hydroxypropyl-β-cyclodextrin inclusion compound. The present invention uses hydroxypropyl-β-cyclodextrin to include maackiain. Compared with parent β-cyclodextrin, hydroxypropyl-β-cyclodextrin has higher water solubility, lower toxicity and excellent inclusion properties. Its stability and solubility can be significantly improved after inclusion with maackiain. The preparation method provided by the invention has simple steps and sufficient inclusion.

The present invention provides a maackiain-hydroxypropyl-β-cyclodextrin inclusion compound prepared by the preparation method described in the above solutions. Molecular docking simulation shows that maackiain enters from the small opening end of hydroxypropyl-β-cyclodextrin and penetrates into the cavity of hydroxypropyl-β-cyclodextrin; the results of the examples show that after the inclusion compound is formed, the solubility of maackiain increased from 0.35 mg·mL⁻¹ to 3.2 mg·mL⁻¹, and the stability was also significantly improved.

### BRIEF DESCRIPTION OF DRAWINGS

FIG. 1 is the 1H NMR spectrum of HPβCD and the MAA/HPβCD inclusion compound in Example 1 of the present invention;

FIG. 2 is the ROESY spectrum of the MAA/HPβCD inclusion compound in D₂O in Example 1 of the present invention;

FIG. 3 is the inclusion model of MAA and HPβCD;

FIG. 4 shows the XRD patterns of MAA, HPβCD, the physical mixture of MAA and HPβCD, and the MAA/HPβCD inclusion compound in Example 1 of the present invention;

FIG. 5 shows the SEM images of MAA, HPβCD, the physical mixture of MAA and HPβCD and the MAA/HPβCD inclusion compound in Example 1 of the present invention;

FIG. 6 is the DSC patterns of MAA, HPβCD, the physical mixture of MAA and HPβCD, and the MAA/HPβCD inclusion compound in Example 1 of the present invention;

FIG. 7 is the curve of relative absorbance vs time of MAA and MAA/HPβCD inclusion compound in Example 1 of the present invention at pH=7.6 and pH=1.5;

FIG. 8 is the Job curve of MAA/HPβCD inclusion compound in pH=3.0 buffer solution in Example 1 of the present invention;

FIG. 9 is the UV absorption curve of MAA in the buffer solution/ethanol (V/V=4:1) of pH=3.0 and pH=10.5 in Example 1 of the present invention;

FIG. 10 is the optimal conformation of molecule docking of the MAA/HPβCD inclusion compound according to the present invention.

### DETAILED DESCRIPTION OF THE INVENTION

The present application provides a method for preparing a maackiain-hydroxypropyl-β-cyclodextrin inclusion compound, comprising the following steps:
adding water and hydroxypropyl-β-cyclodextrin to a maackiain solution in sequence, and stirring the mixture in darkness to obtain a maackiain-hydroxypropyl-β-cyclodextrin inclusion compound;
the solvent of the maackiain solution is an organic solvent.

In the present invention, the structural formula of the maackiain (MAA) is as shown in Formula I:

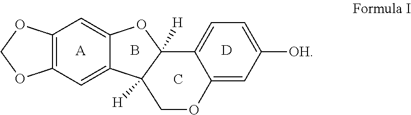

In the present invention, the organic solvent preferably includes one or more of methanol, ethanol, isopropyl alcohol, acetonitrile and acetone, more preferably ethanol; the concentration of the maackiain solution is preferably 2.125 to 4.25 mg/mL, more preferably 3 mg/mL; in the present invention, it is preferred to mix maackiain and an organic solvent and then stir the mixture at room temperature until completely dissolved; the stirring time is preferably 24 h.

After obtaining the maackiain solution, the present invention sequentially adds water and hydroxypropyl-β-cyclodextrin to the maackiain solution. In the present invention, the volume ratio of the maackiain solution to water is preferably 2-4:8-10; the water is used to dissolve hydroxypropyl-β-cyclodextrin and serves as a solvent for the inclusion reaction. In a specific embodiment of the present invention, it is preferable to observe the maackiain solution while adding water. In the present invention, it is preferable to add water until the maackiain solution just becomes turbid. At that time, the maackiain reaches the critical state of precipitation, thereby ensuring that hydroxypropyl-β-cyclodextrin and maackiain can be included in sufficient solvents.

In the present invention, the structural formula of the hydroxypropyl-β-cyclodextrin (HPβCD) is as shown in Formula II, and its simplified structural formula is as shown in Formula III:

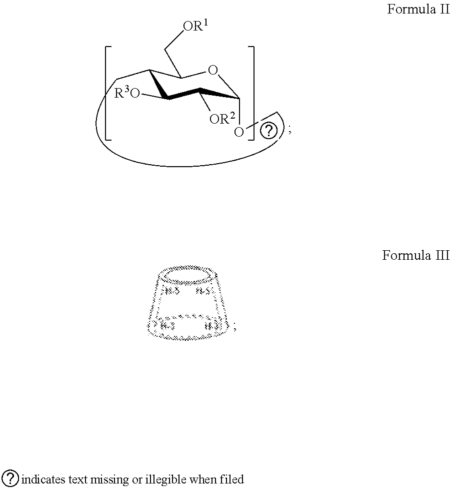

In formula II, R¹, R³ are H, R² is —CH₂CH(CH₃)OH (hydroxypropyl).

In the present invention, the molar ratio of maackiain to hydroxypropyl-β-cyclodextrin in the maackiain solution is preferably 1:1 to 1:2, more preferably 1:1. The present invention has no special requirements on the sources of maackiain and hydroxypropyl-β-cyclodextrin, and the above-mentioned substances available on the market can be used.

After adding water and hydroxypropyl-β-cyclodextrin, the present invention stirs the mixture in darkness. In the present invention, the stirring in darkness preferably lasts 1 h to 5 h, more preferably 2 h to 3 h; the present invention has no special requirements for the stirring speed, as long as it can ensure that maackiain and hydroxypropyl-β-cyclodextrin can be included in a uniformly mixed state. In the present invention, stirring is carried out in darkness to avoid the decomposition of maackiain.

After stirring in darkness is completed, the present invention preferably carries out filtration, rotary evaporation and drying of the resulting stirred liquid in sequence to obtain the inventive maackiain-hydroxypropyl-β-cyclodextrin inclusion compound (MAA/HPβCD inclusion compound). In the present invention, it is preferred to use a 0.45 μm microporous filter membrane for filtration. In the present invention, unreacted maackiain is removed by filtration.

In the present invention, the temperature of the rotary evaporation is preferably 55-70° C., more preferably 60° C.; in the present invention, the filtrate obtained by filtration is preferably rotary evaporated to dryness.

The present invention has no special requirements for the temperature and time of the drying, as long as the moisture in the residue from rotary evaporation can be completely removed.

The present invention provides a maackiain-hydroxypropyl-β-cyclodextrin inclusion compound prepared by the preparation method described in the above solutions. Molecular docking simulation shows that maackiain enters from the small opening end of hydroxypropyl-β-cyclodextrin and penetrates into the cavity of hydroxypropyl-β-cyclodextrin, thereby forming the inclusion compound of the present invention. After the inclusion compound is formed, the stability and solubility of maackiain are significantly improved, so that it has broad application prospects.

A maackiain-hydroxypropyl-β-cyclodextrin inclusion compound provided by the invention and a preparation method thereof will be described in detail below in conjunction with examples, but they cannot be understood as restrictions on the scope of protection of the present invention.

### Example 1

### I. Raw Materials and Instruments

Maackiain (C₁₆H₁₂O₅, FW=284.27, purity >98%), supplied by Xinyi Technology, Weibin District, Baoji City; 2-hydroxypropyl-β-cyclodextrin (HPβCD, FW=1460), purchased from ABCR GmbH & Co. KG. The experimental water was commercially available ultrapure water, and the remaining reagents were commercially available and of analytical grade.

UV-8453 ultraviolet-visible spectrometer, Agilent Technology Co., Ltd.; 85-2 thermostatic magnetic stirrer, Jiangsu Guohua Instrument Co., Ltd.; BSA224S-CW analytical balance, Sartorius; FEI PHENO scanning electron microscope (SEM), JEOL Ltd.; D/max-3B X-ray powder diffractometer, Rigaku Corporation, Japan; STA449F3 thermogravimetric analyzer, NETZSCH GmbH, Germany.

### II. Preparation Steps

Preparation of maackiain-hydroxypropyl-β-cyclodextrin inclusion compound: 8.5 mg of maackiain was accurately weighed according to a molar ratio of 1:1, added to a 25 mL round bottom flask to mix with 2 mL of ethanol, stirred at room temperature for 1 day, until the sample was completely dissolved. A certain amount of water was added until the solution in the round-bottomed flask just became turbid, then 43.8 mg of HPβCD was added and the mixture was stirred in darkness for 1 h. The system was filtered to remove unreacted MAA, and the filtrate was then filtered with a 0.45 μm microporous filter membrane, rotarily evaporated to dryness at 60° C., and dried under vacuum to obtain an MAA/HPβCD inclusion compound.

At the same time, a physical mixture of maackiain and hydroxypropyl-β-cyclodextrin was prepared as a control: MAA and HPβCD were weighed at a molar ratio of 1:1, and mixed thoroughly.

### III. Characterization of Inclusion Compound

### (1) NMR Analysis

D₂O was used as a solvent to dissolve HPβCD and the MAA/HPβCD inclusion compound respectively, and 1H NMR and 2D NMR spectra were measured.

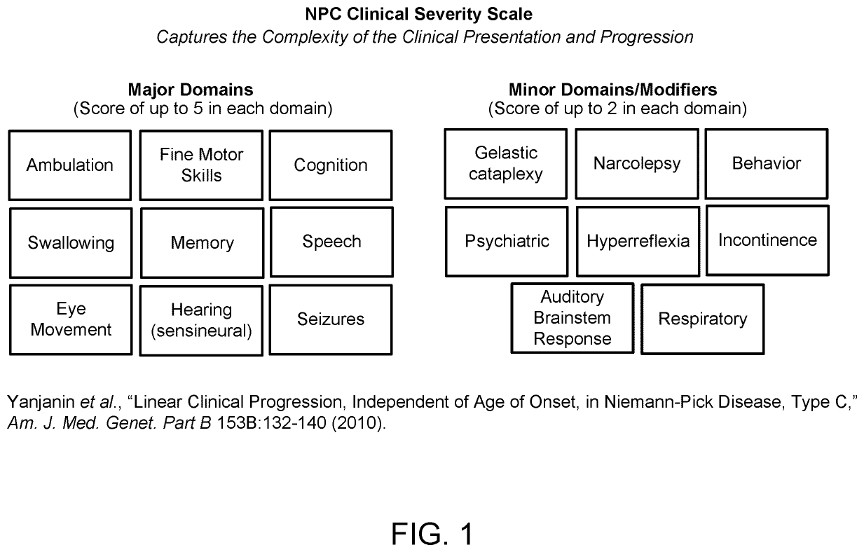

FIG. 1 shows the 1H NMR spectra of HPβCD and the MAA/HPβCD inclusion compound, where (a) is the 1H NMR spectrum of HPβCD, (b) is the 1H NMR spectrum of the MAA/HPβCD inclusion compound, and * in FIG. 1 is the marker of the water peak.

The ¹H NMR chemical shift values of HPβCD and the MAA/HPβCD inclusion compound are shown in Table 1;

TABLE 1
The ¹HNMR chemical shift values of HPβCD
and the MAA/HPβCD inclusion compound

Chemical shift δ (ppm)

| | | HPβCD | MAA/HPβCD |
|---|---|---|---|
| H-1 | d | 4.99 | 4.95 |
| H-2 | dd | 3.54 | 3.53 |
| H-3 | dd | 3.87 | 3.90 |
| H-4 | dd | 3.49 | 3.47 |
| H-5 | m | 3.77 | 3.71 |
| H-6 | dd | 3.77 | 3.63 |
| H-Me | d | 1.03 | 1.05 |

According to FIG. 1, it can be seen that after HPβCD forms an inclusion compound with MAA, its proton chemical shift has changed significantly (see Table 1), the chemical shifts of the H-1, H-2, H-3, H-4, H-5 and H-6 protons have changed by 0.04 ppm, 0.01 ppm, 0.03 ppm, 0.02 ppm, 0.06 ppm, and 0.04 ppm respectively, while the H-3 and H-5 protons are all located in the cyclodextrin cavity, the H-3 proton is located at the large opening end of the cyclodextrin cavity, and the H-5 proton is located at the small opening end of the cyclodextrin cavity. The chemical shift value of the H-5 proton changes greatly, indicating that when MAA enters the HPβCD cavity, it affects the H-5 protons most significantly, showing that MAA enters the cavity from the small opening end.

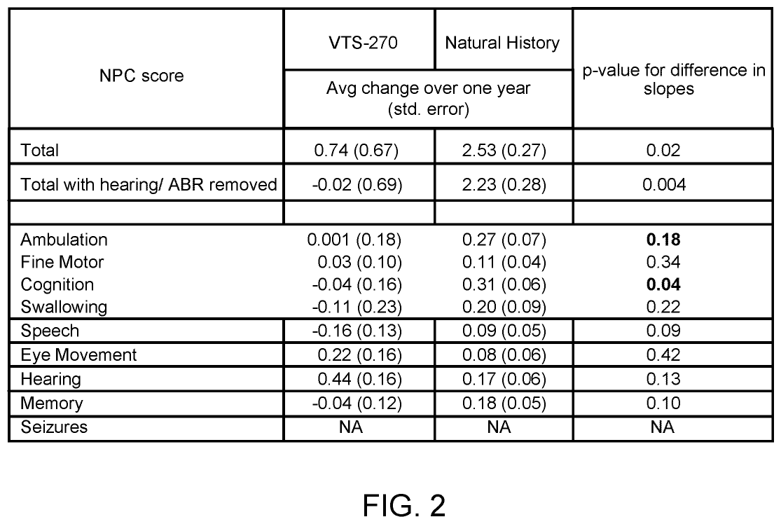

The ROESY spectra of MAA and the MAA/HPβCD inclusion compound are as shown in FIG. 2; FIG. 2 shows that the H-5, H-6, H-8, H-11, H-14 protons of MAA are respectively related to the H-5 and H-6 protons of HPβCD related, indicating that MAA enters the cyclodextrin cavity as a D ring.

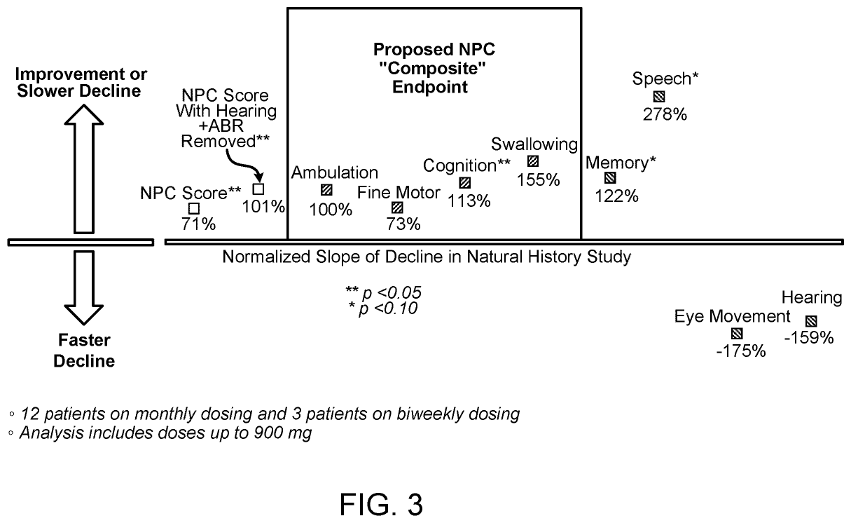

In combination with the analysis results provided by ¹H NMR and ROESY, it shows that the present invention successfully prepares the MAA/HPβCD inclusion compound, and MAA enters from the small opening end of HPβCD as a D ring. The possible inclusion mode of MAA and HPβCD is shown in FIG. 3.

### (2) Powder X-Ray Diffraction (XRD) Analysis

MAA, HPβCD, the MAA/HPβCD inclusion compound, and the physical mixture of)

MAA and HPβCD were respectively pressed uniformly on glass plates under the following experimental conditions for measurement: CuKα (K=1.5460 Å), 40 kV, 40 mA, scanning rate 6°·min⁻¹, scanning step 0.02° (2θ scanning range, 3° to) 50°.

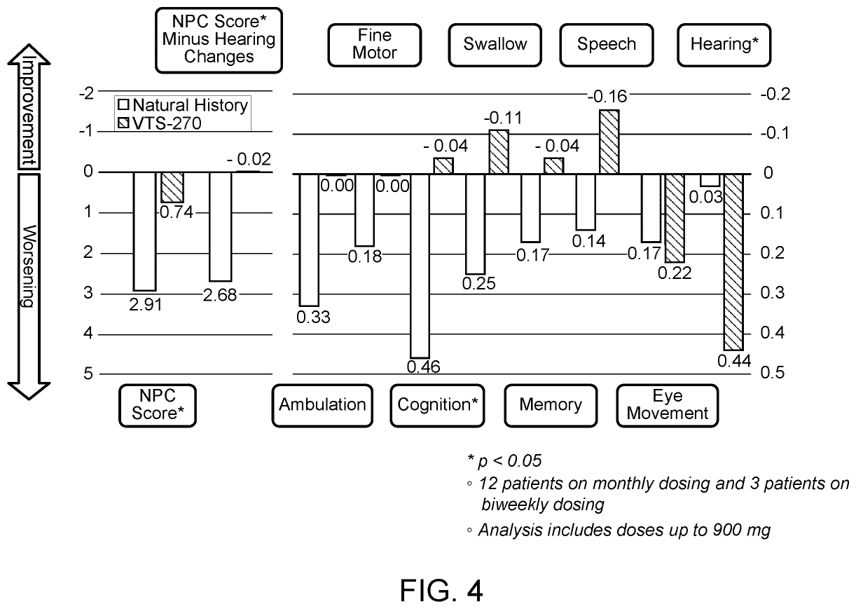

The obtained XRD spectra are as shown in FIG. 4. In FIG. 4, (a) is the XRD spectrum of MAA; (b) is the XRD spectrum of HPβCD; (c) is the XRD spectrum of MAA and HPβCD physical mixture; (d) is the XRD spectrum of the MAA/HPβCD inclusion compound.

It is found from the measurement results that in FIG. 4(a), MAA has a crystalline structure, and the obvious characteristic peak appears at 20:23.00°, in FIG. 4(b), HPβCD has an amorphous structure, and the obvious characteristic peak appears at 2θ:15.00°-35.00°, in FIG. 4(c), the physical mixture of MAA/HPβCD has the diffraction peak of MAA and the amorphous structure of HPβCD; in FIG. 4(d), the peak shape and intensity of the MAA/HPβCD inclusion compound are obviously different and in halo-like amorphous form, and the characteristic peak appears at 2θ:16.00°-21.00°. The analysis of the results shows that the crystal forms of the diffraction peaks of MAA and HPβCD change significantly before and after inclusion. This result further demonstrates that MAA and HPβCD form an inclusion compound.

### (3) Determination by Scanning Electron Microscope

The scanning electron microscope images of MAA, HPβCD, the physical mixture of

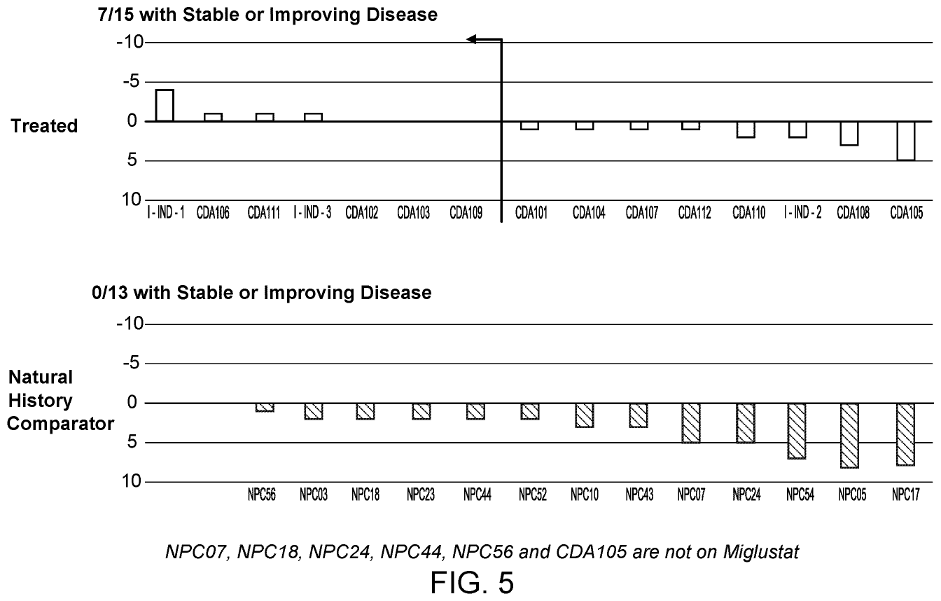

MAA and HPβCD, and the MAA/HPβCD inclusion compound are as shown in FIG. 5, wherein (a) is the SEM image of MAA; (b) is the SEM image of HPβCD; (c) is the SEM image of the physical mixture of MAA and HPβCD; (d) is the SEM image of the MAA/HPβCD inclusion compound.

As can be seen from FIG. 5, MAA (FIG. 5a) is flake-shaped, HPβCD (FIG. 5b) is a spherical crystal, and the physical mixture of MAA and HPβCD (FIG. 5c) is the simple stacking of the two and there exist flake and spherical crystals, and the structure of the inclusion compound (FIG. 5d) is in the form of a mass and slightly irregular. These significant changes indicate that HPβCD and MAA have formed an inclusion compound.

### (4) Thermal Analysis

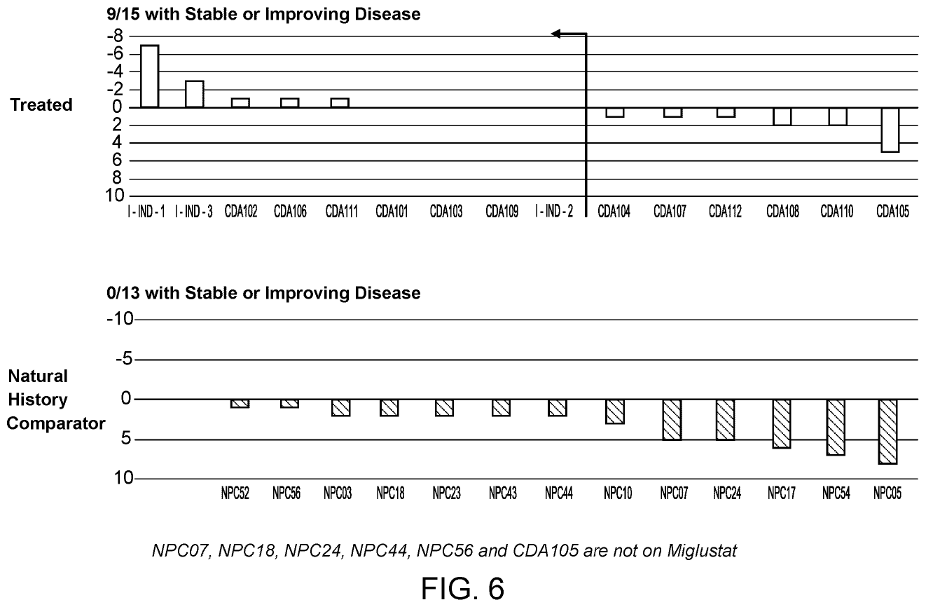

9.0 mg of MAA, 8.0 mg of HPβCD, and 6.0 mg of the MAA/HPβCD inclusion compound were weighed, respectively. Under N₂ protection, with a flow rate of 70 mL·min⁻¹, at a rate of 10° C.·min⁻¹, the temperature was raised from 40° C. to 500° C., and differential scanning calorimetry (DSC) was carried out. The DSC curve was measured, as shown in FIG. 6. In FIG. 6, (a) is the DSC curve of MAA; (b) is the DSC curve of HPβCD; (c) is the DSC curve of the physical mixture of MAA and HPβCD; (d) is the DSC curve of the MAA/HPβCD inclusion compound.

As can be seen from FIG. 6, MAA has an endothermic peak at 110° C. and 181.2° C. respectively, HPβCD has a broad endothermic peak at 99° C., and the physical mixture of MAA and HPβCD still has the endothermic peak of HPβCD, but with weakened peak intensity. After the inclusion compound is formed, the endothermic peaks of MAA and HPβCD disappear, and a new endothermic peak appears at 330.4° C. The DSC curve analysis results show that MAA and HPβCD form an inclusion compound.

### (5) Water Solubility Experiment

The solubility of MAA and the MAA/HPβCD inclusion compound in water was determined by saturated solution method. 6.0 mg of MAA and 9.8 mg of the MAA/HPβCD inclusion compound were added to 2 mL of distilled water, and stirred for 4 h in darkness at the temperature of 20±2° C. Then the mixture was filtered with cellulose acetate membrane (0.45 μm); the filtrate was distilled under reduced pressure and dried to obtain a white solid.

As calculated, the solubility of MAA is 0.35 mg mL⁻¹, and the solubility of MAA in the MAA/HPβCD inclusion compound is 3.2 mg·mL⁻¹.

### (6) Stability Experiment

Four brown volumetric flasks of 25 mL were taken, a certain volume of MAA solution was added to each of them, then an appropriate amount of HPβCD was added to another 2 bottles. Water-ethanol mixture (V/V=4:1) and a buffer solution of pH=1.5 (simulating human gastric juice) and pH=7.6 (simulating human intestinal juice) were added for dilution to a constant volume. After standing in a thermostatic water bath at 37° C. for 1 h, absorbance was measured at a maximum wavelength of 310 nm every (12±2 h) and the relative absorbance A/A₀ was calculated (where A is the absorbance measured every 12±2 h, and A₀ is the original absorbance). All experiments were measured three times in parallel.

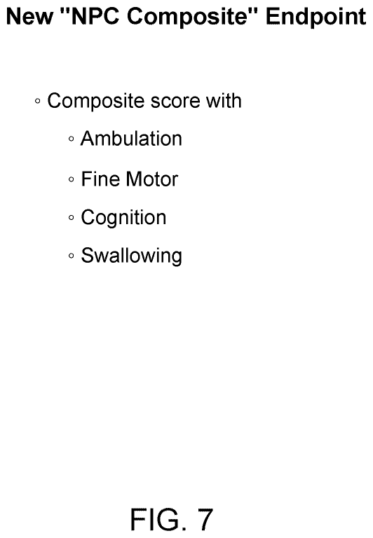

The results are as shown in FIG. 7. In FIG. 7, (a) is the curve of relative absorbance vs time of MAA at pH=7.6, and (b) is the curve of relative absorbance vs time of the MAA/HPβCD inclusion compound at pH=7.6, (c) is the curve of relative absorbance vs time of MAA at pH=1.5, (d) is the curve of relative absorbance vs time of the MAA/HPβCD inclusion compound at pH=1.5.

As can be seen from FIG. 7, at pH=7.6 and pH=1.5, the absorbance of MAA increases with time with larger fluctuation, and the ultraviolet absorption decreases with the increase of time by approximately 70% and 60% respectively, which indicates that MAA will slowly decompose to a certain extent, and the UV absorption reduction trend of the inclusion compound in the two environments is significantly smaller than that of the MAA solution. From the analysis of the results in FIG. 7, it can be seen that after MAA is included by HPβCD, its stability is significantly improved regardless of acidic or alkaline conditions.

### (7) Spectral Analysis

### (a) Job Curve Determination

The stoichiometry of MAA/HPβCD can be determined by the Job method. A series of mixed solutions of MAA and HPβCD were prepared by using a buffer solution with pH=3.0, the total concentration of MAA and HPβCD was kept at 7.2×10⁻⁵ mol·L⁻¹, where the molar ratio of MAA to HPβCD changed between 0 and 1. The absorbance was measured at its maximum absorption wavelength of 310 nm.

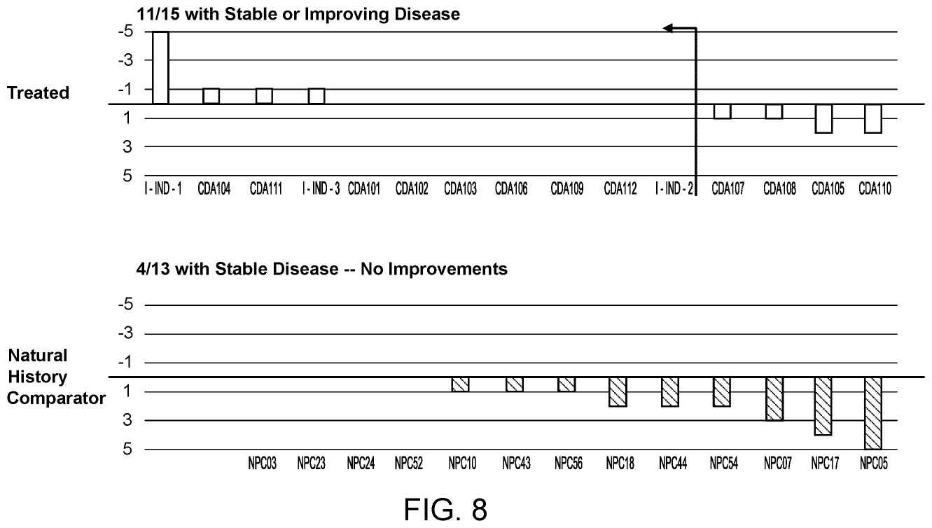

The Job curve is shown in FIG. 8. The abscissa corresponding to the highest point of the curve is 0.5, from which it can be judged that the stoichiometric ratio of the inclusion compound is 1:1.

### (b) UV Spectrum Measurement

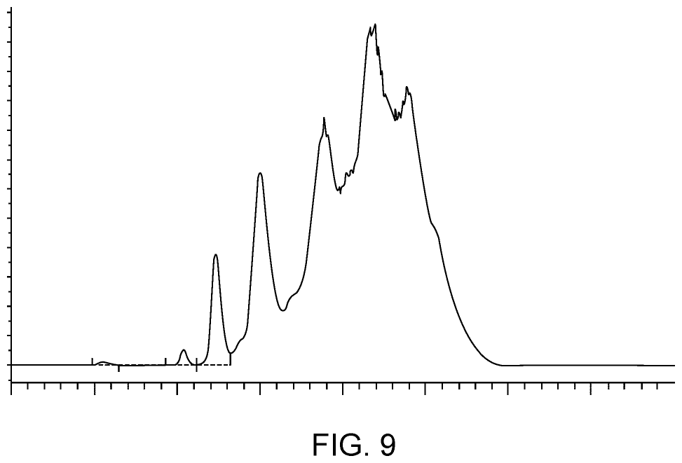

The concentration of MAA was kept at 0.072 mmol·L⁻¹, HPβCD solutions were prepared according to the concentration gradients in Table 2 sequentially. The solution was diluted to volume using a buffer solution of pH=3.0 or pH=10.5 and ethanol (V/V=4:1) as the solvent, allowed to stand for 30 min, then measured for absorbance. By observing the law of change of the absorbance of the medicine liquid with the concentration of HPβCD, and the UV spectrum of the host-guest inclusion compound, all experiments were measured three times in parallel, and the resulting UV spectrum is shown in FIG. 9. In FIG. 9, A is the UV absorption curve of MAA in a buffer solution of pH=3.0/ethanol (V/V=4:1), and B is the UV absorption curve of MAA in a buffer solution of pH=10.5/ethanol (V/V=4:1); a-i in FIG. 9 respectively correspond to the concentration points in Table 3.

TABLE 2
The concentration gradient of hydroxypropyl-β-cyclodextrin (HPβCD) in
ultraviolet-visible spectrum titration under pH = 3.0 and pH = 10.5 conditions

| Host | Concentration gradient (mmol/L) | pH |
|---|---|---|
| HPβCD | 0, 0.231, 0.329, 0.471, 0.672, 0.960, 1.372, 1.960, 2.800, 4.000 | 3.0 |
| | 0, 0.231, 0.329, 0.471, 0.672, 0.960, 1.372, 1.960, 2.800, 4.000 | 10.5 |

First, it is assumed that the MAA/HPβCD inclusion compound is formed at an inclusion ratio of 1:1 (Formula 1), and the Ks inclusion constant (Formula 2) can be obtained according to Lambert-Beer's law;

$$MAA + HP β CD MAA / HP β CD \qquad ( 1 )$$

$$Ks = \frac{[ MAA / HP β CD ]}{[ MAA ] [ HP β CD ]} = \frac{Δ A / Δ ε}{( {[ MAA ]}_{0} - Δ A / Δ ε ) ( {[ HP β CD ]}_{0} - Δ A / Δ ε )} \qquad ( 2 )$$

In formula 2, [MAA]₀ and [HPβCD]₀ are original concentrations; [MAA·HPβCD], [MAA] and [HPβCD] are equilibrium concentrations; AA is the change in absorbance of MAA in the presence or absence of HPβCD (A-A₀ or A₀-A), and after forming the supramolecular complex, ΔA can be expressed as ΔA=Δε[MAA·HPβCD], which represents the difference in the molar extinction coefficient of MAA in the presence or absence of HPβCD.

Finally, the inclusion stability constants and Gibbs frcc energy changes of MAA and HPβCD under different pH values can be calculated by the nonlinear least-squares method, as shown in Table 3. The inset in the upper right corner of FIG. 9 shows the curve fitting results of the inclusion coordination of MAA and HPβCD, which reflects a good linear relationship between experimental values and theoretical values, further confirming the previous hypothesis, that is, MAA and HPβCD are included at 1:1.

TABLE 3
The stability constant (Kₛ) and Gibbs free energy change (−ΔG°) when
MAA and HPβCD form an inclusion compound in the mixed solution
of water-ethanol (V:V = 4:1, pH = 3.0, pH = 10.5, 25° C.)

| | | | | Gibbs free energy change |
|---|---|---|---|---|
| Host | pH | Stability Ks /M⁻¹ | Log Ks | −ΔG°/KJ · mol⁻¹ |
| HPβCD | 3.0 | 1029 | 3.0119 | 17.194 |
| | 10.5 | 318 | 2.502 | 14.283 |

### (c) Molecular Docking of MAA and HPβCD

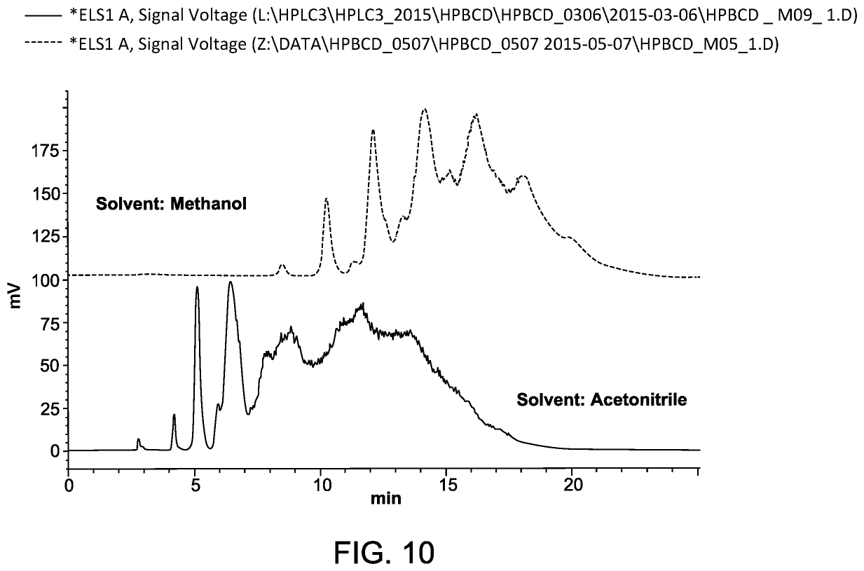

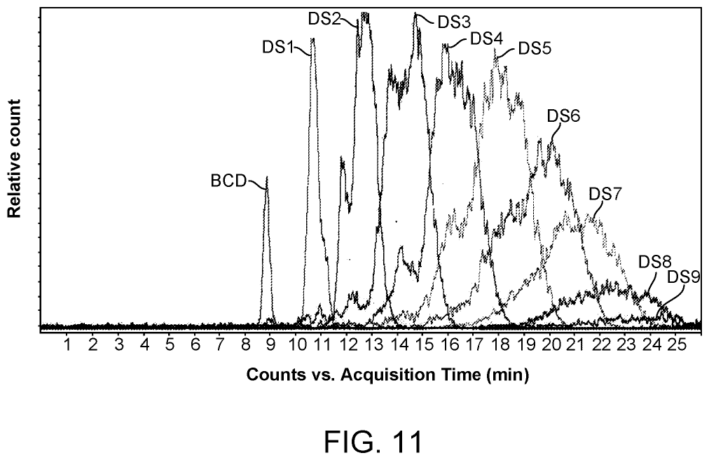

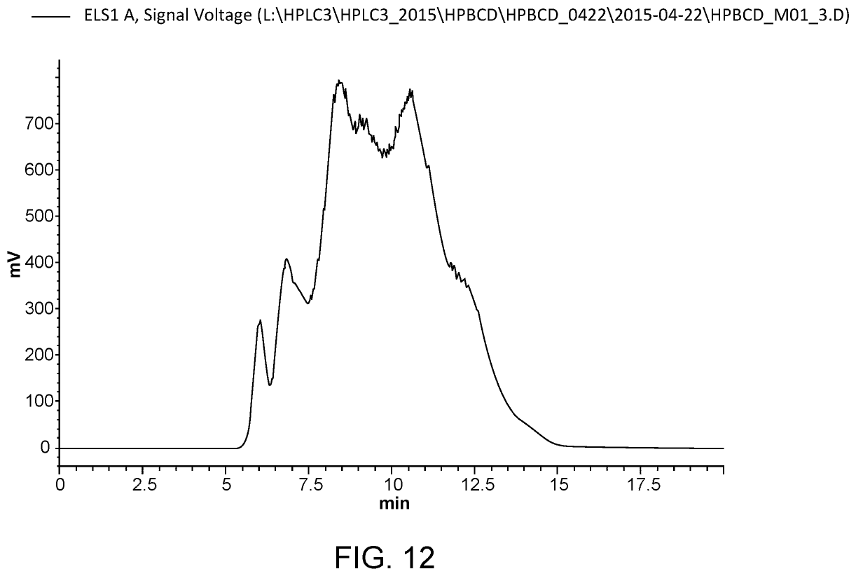

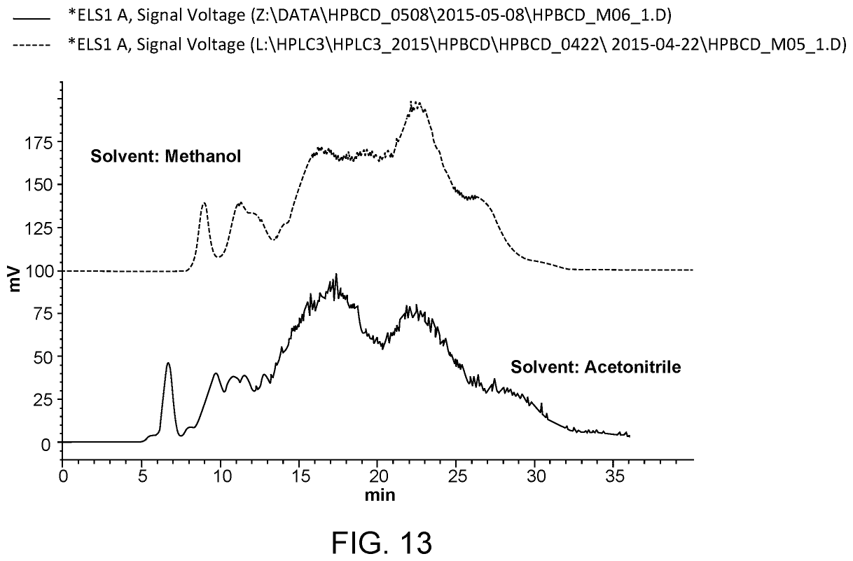

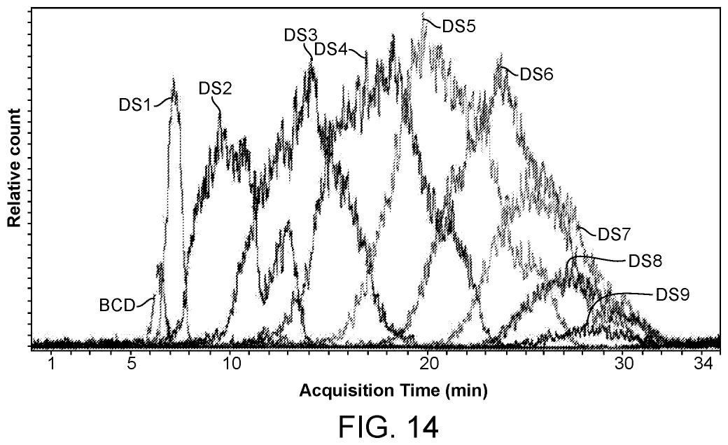

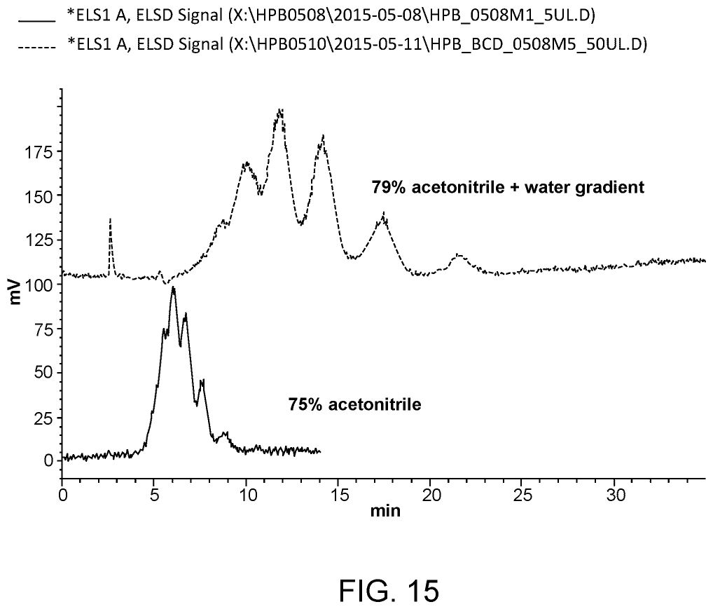

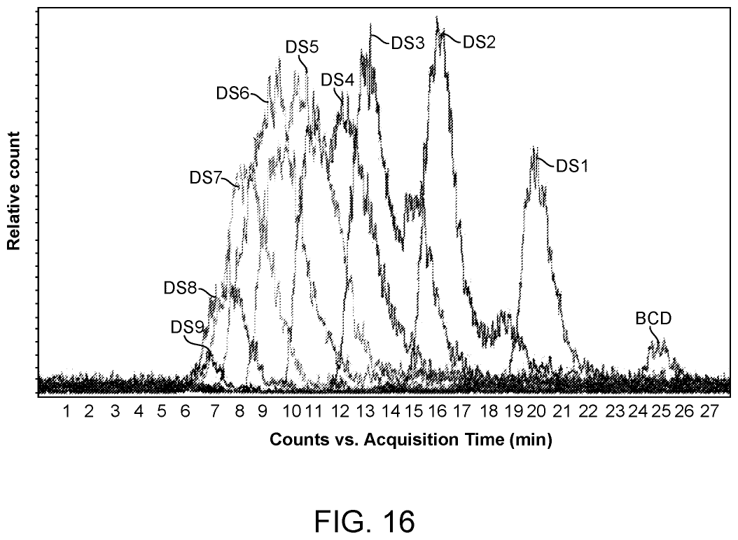

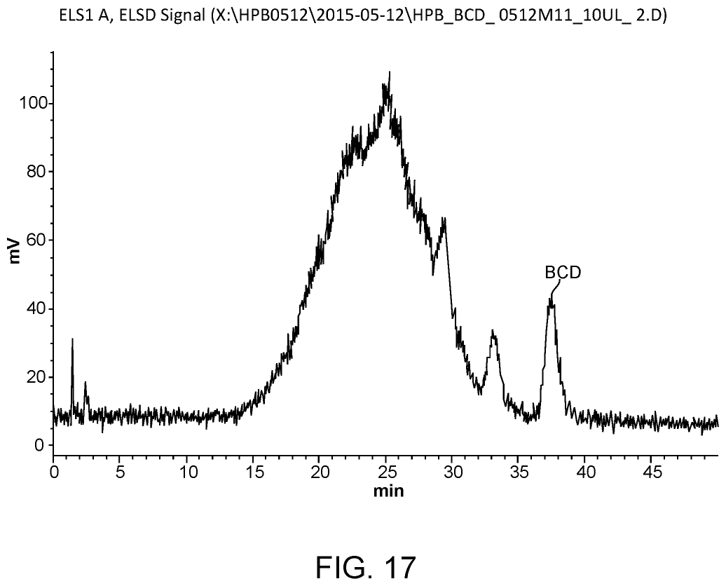

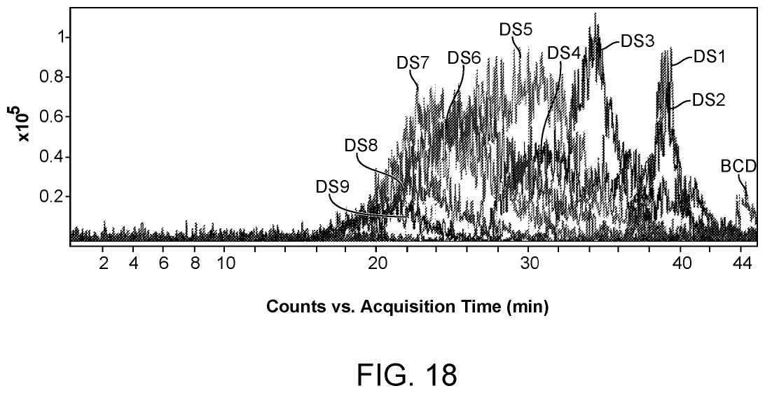

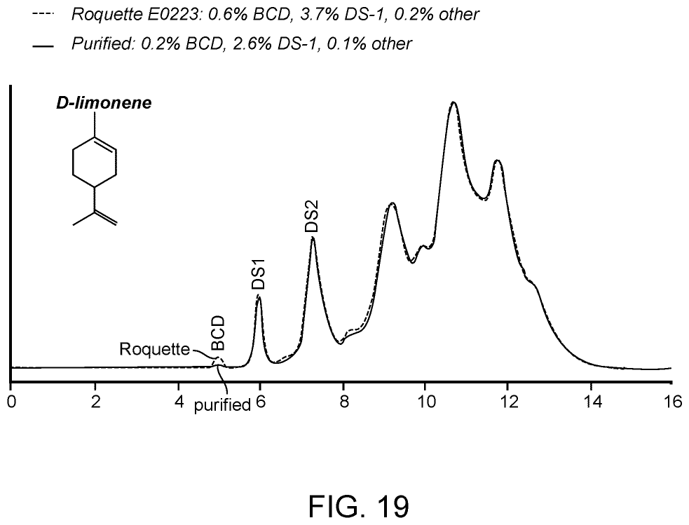

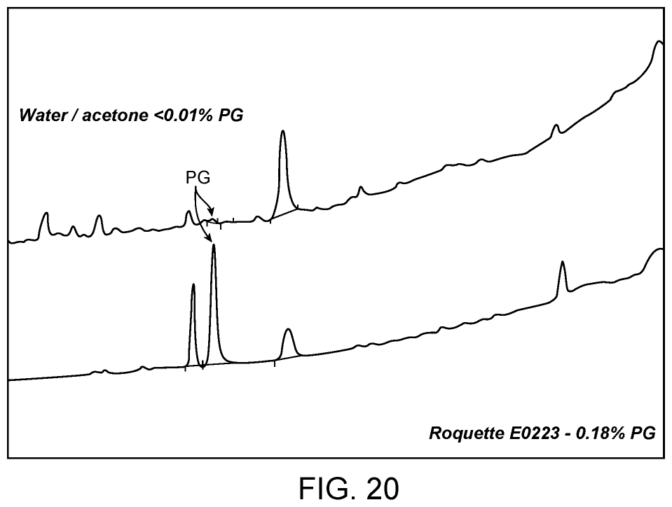

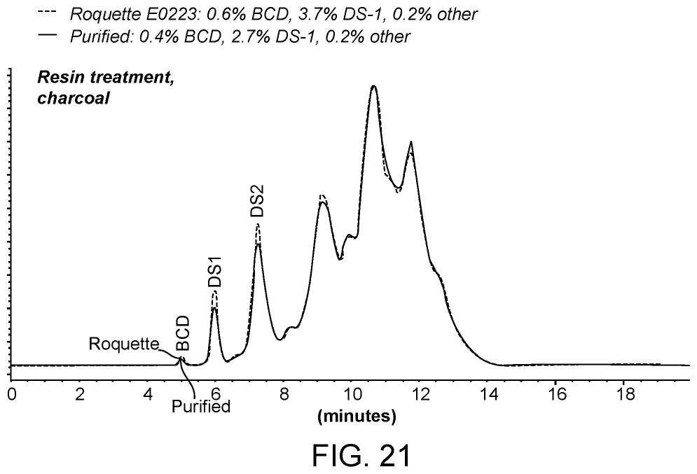

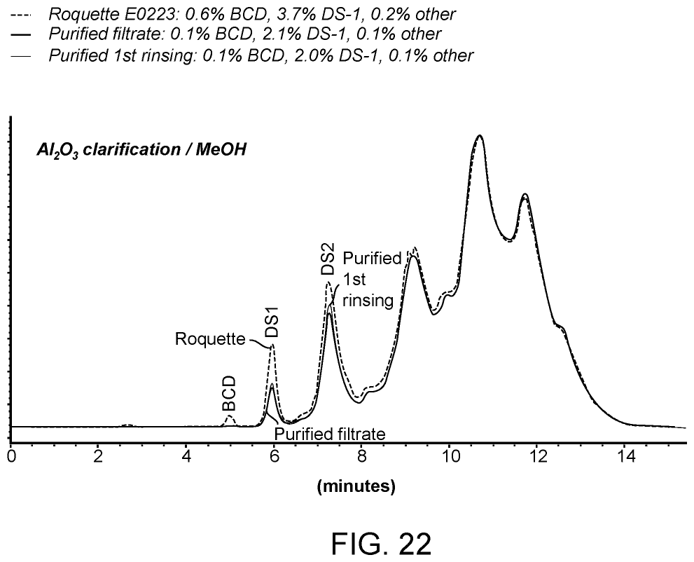

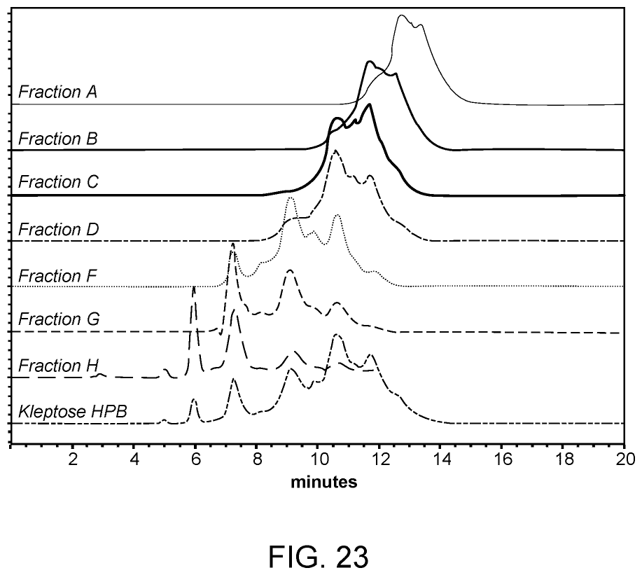

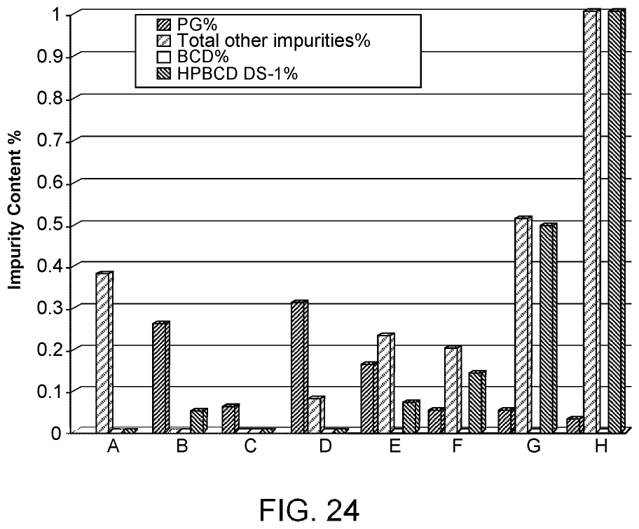

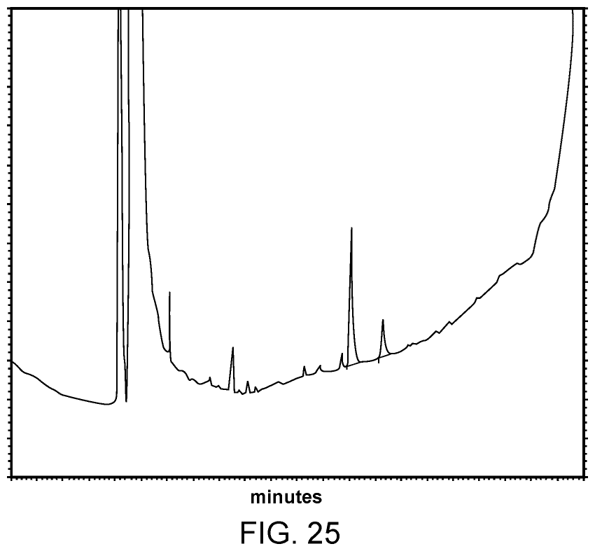

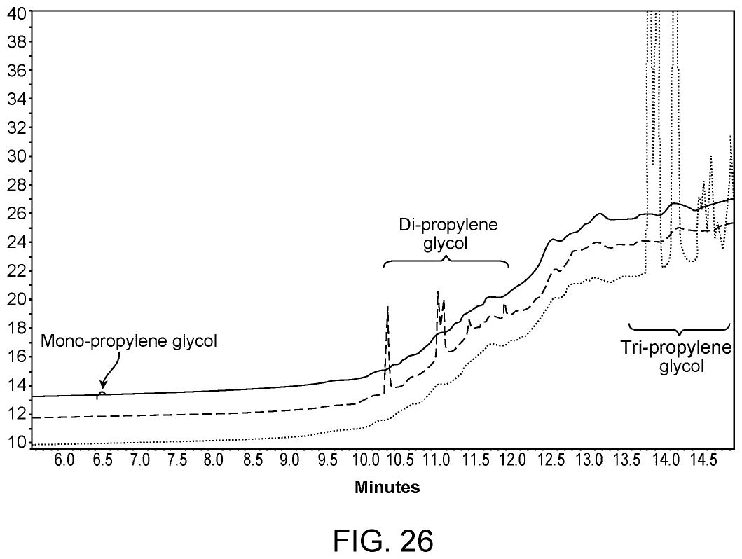

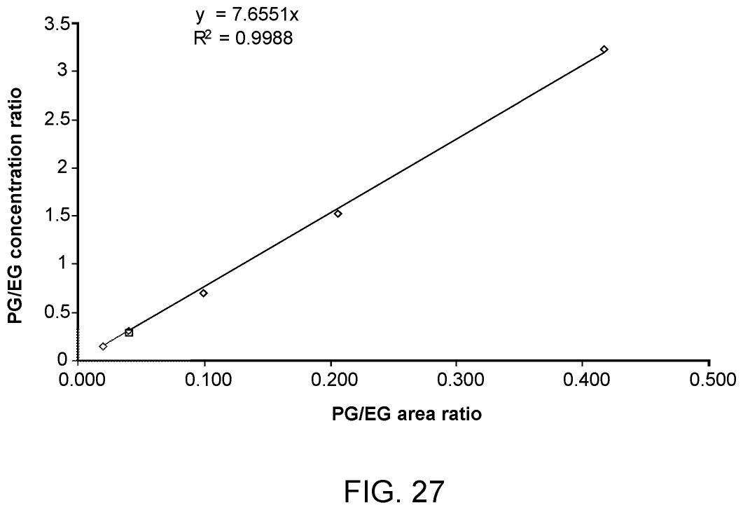

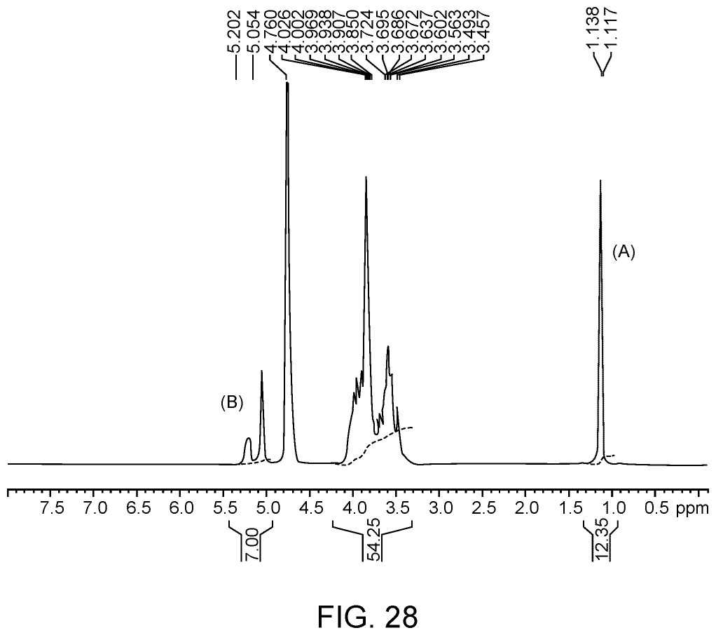

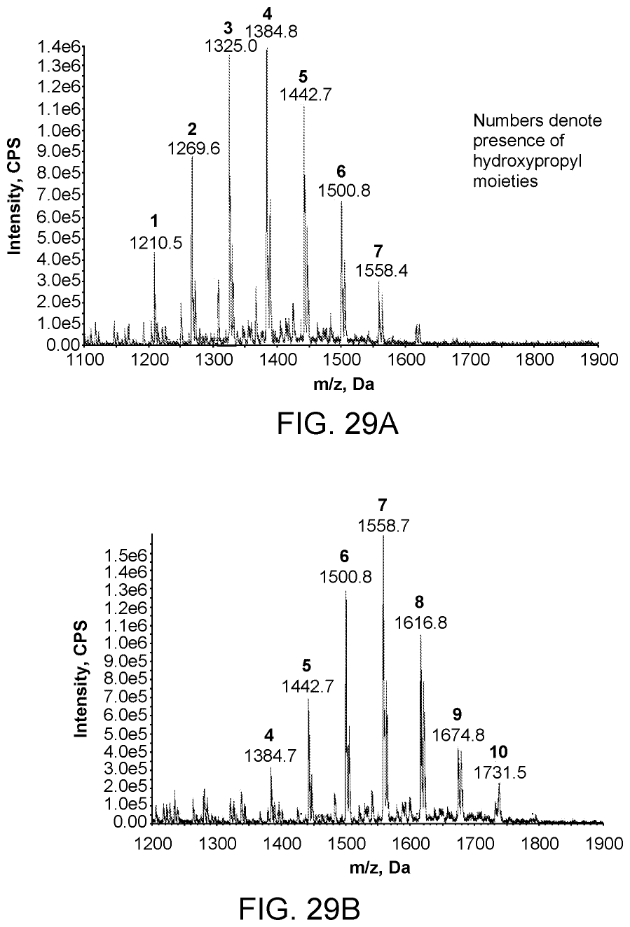

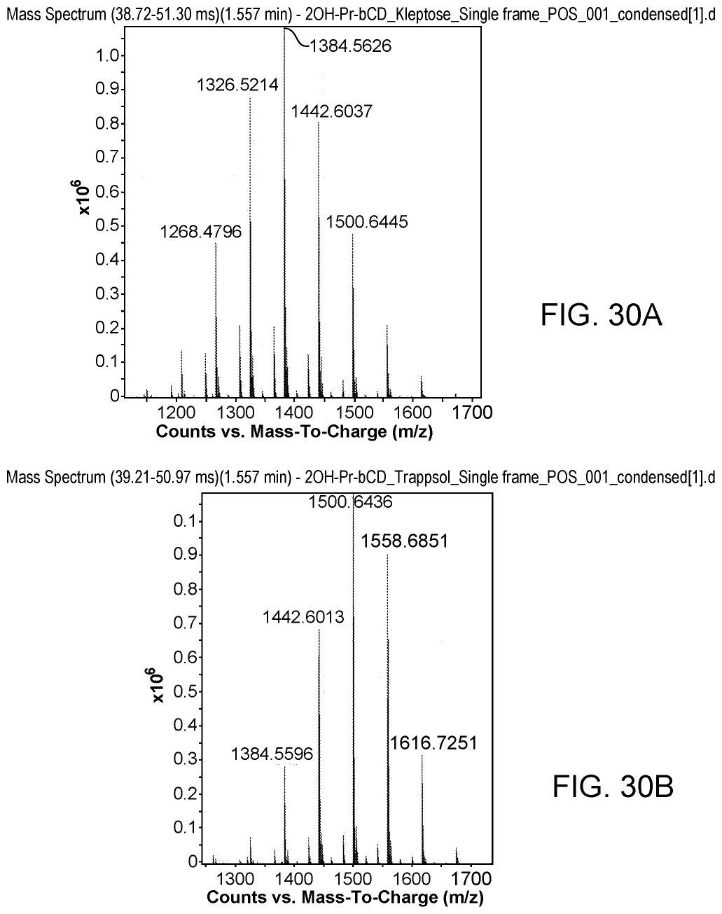

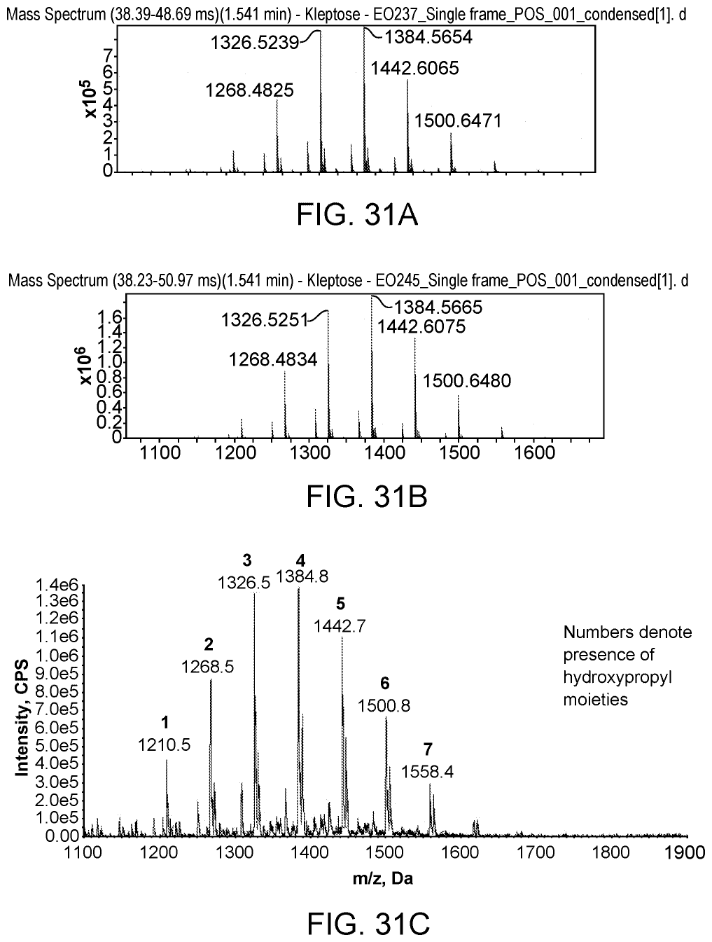

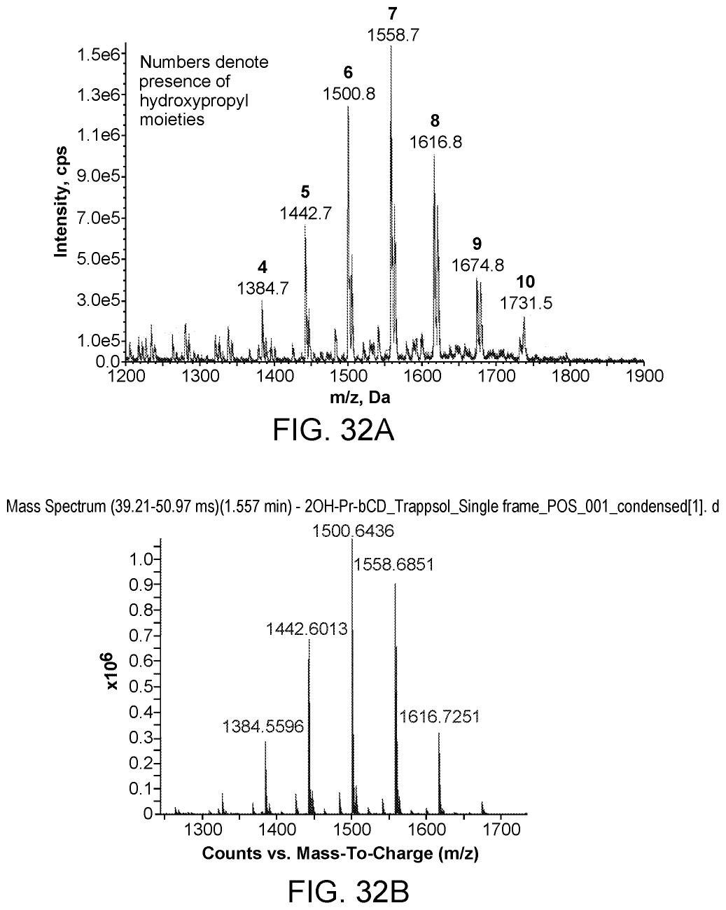

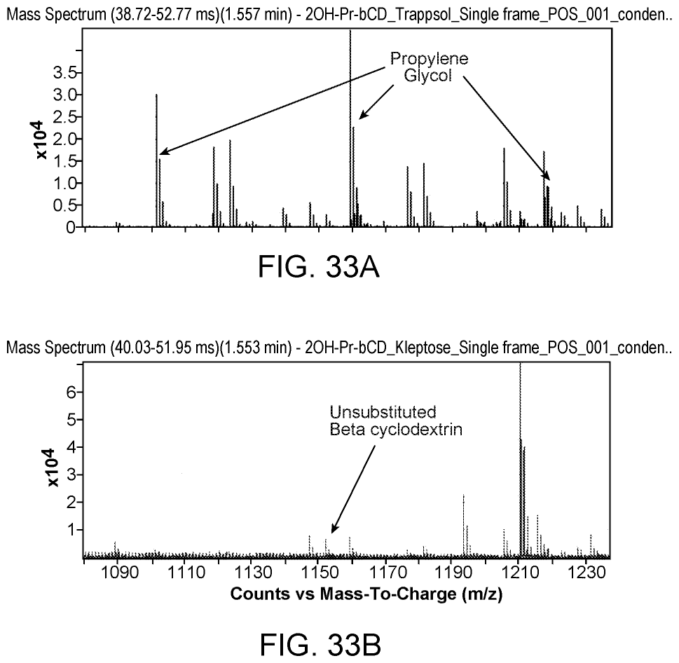

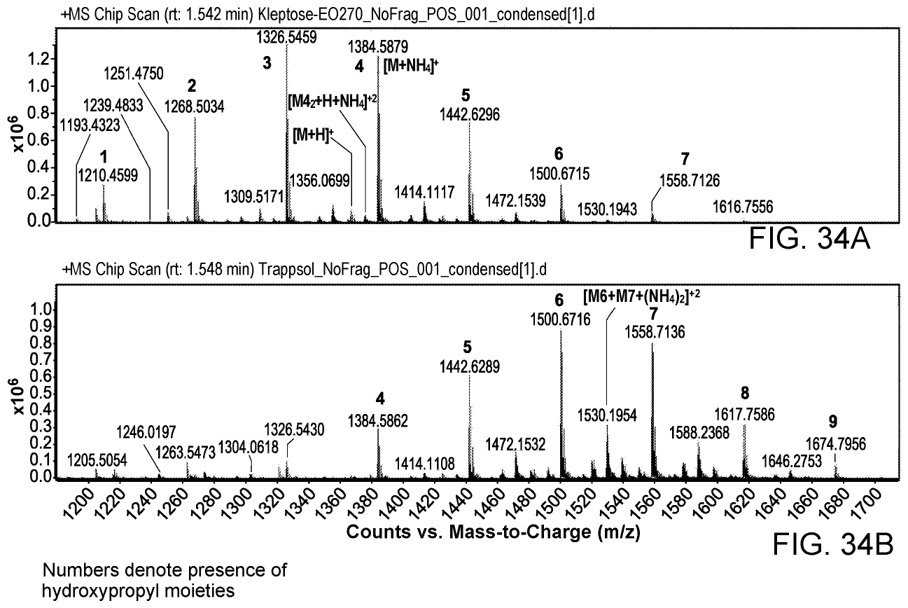

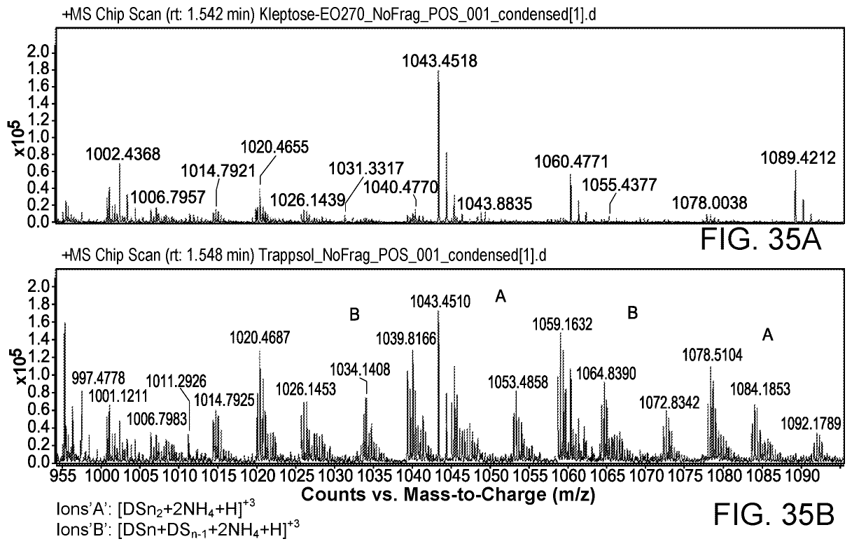

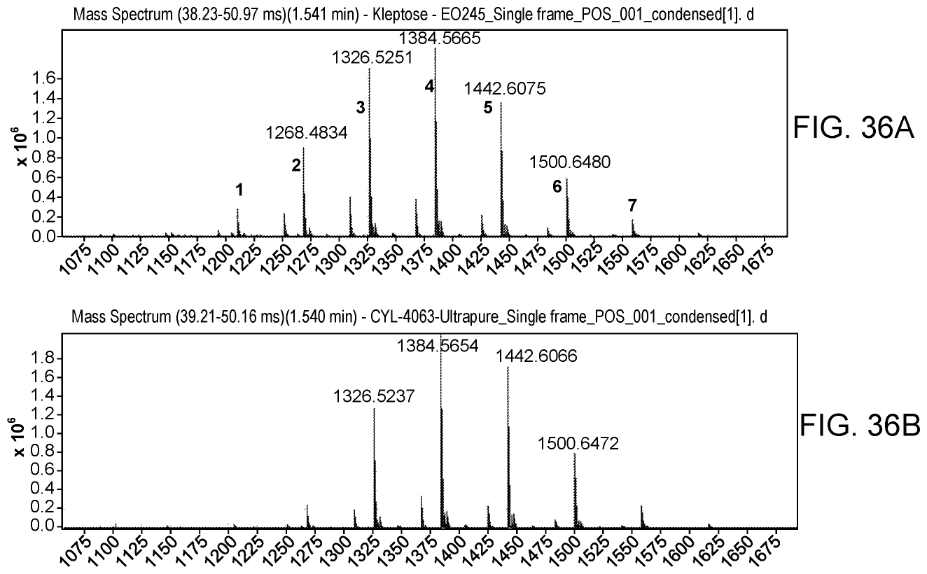

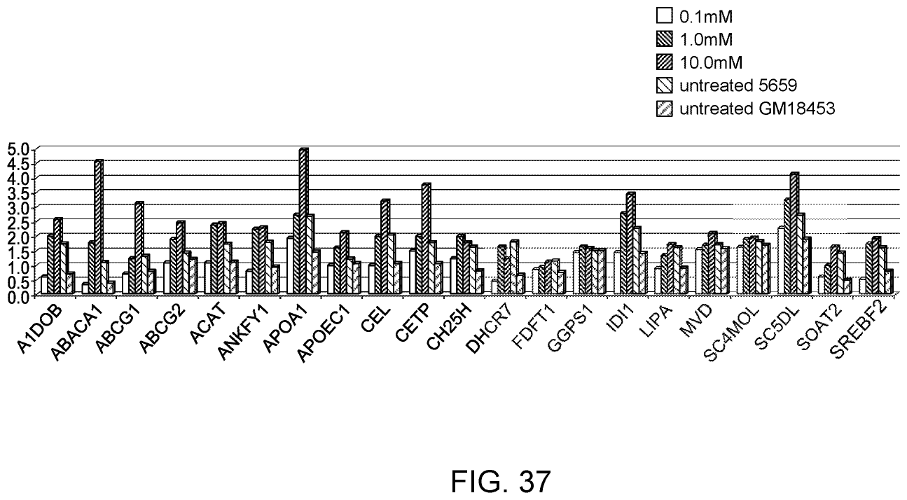

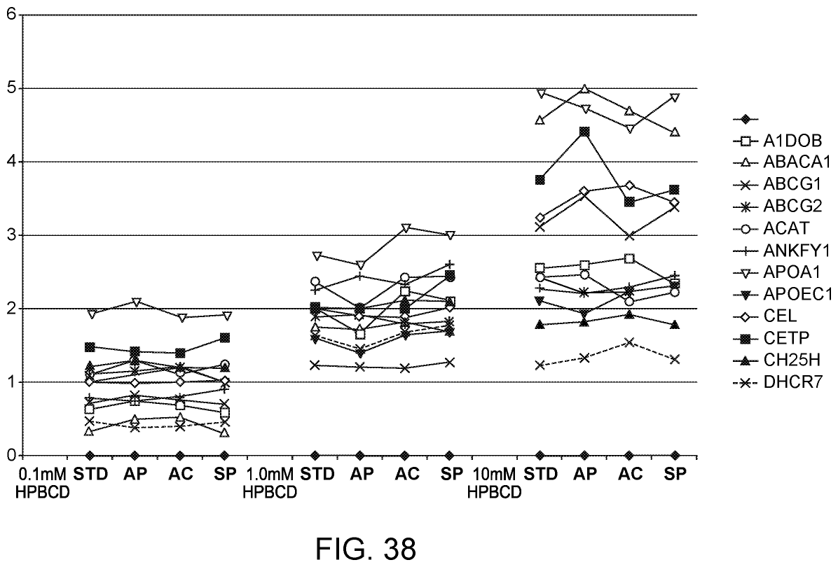

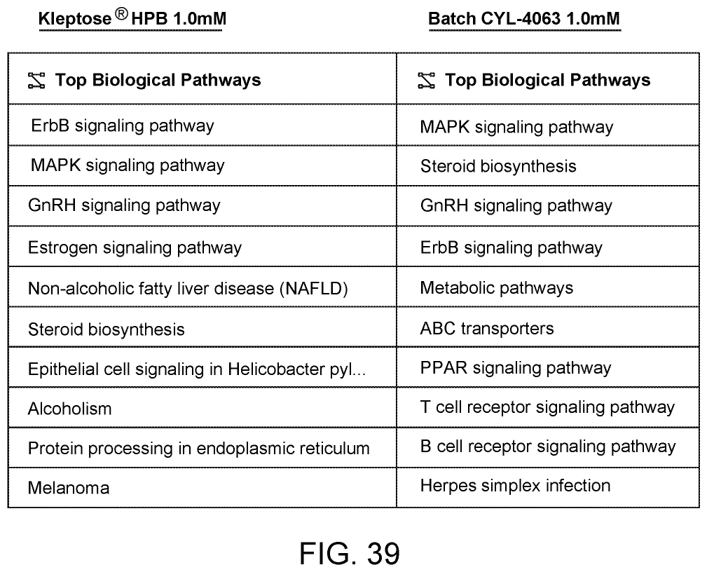

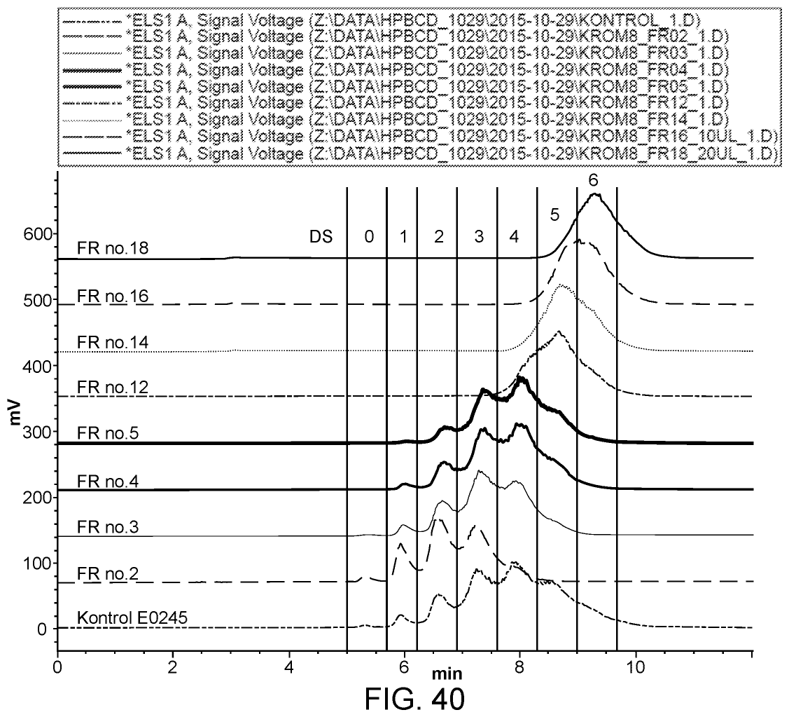

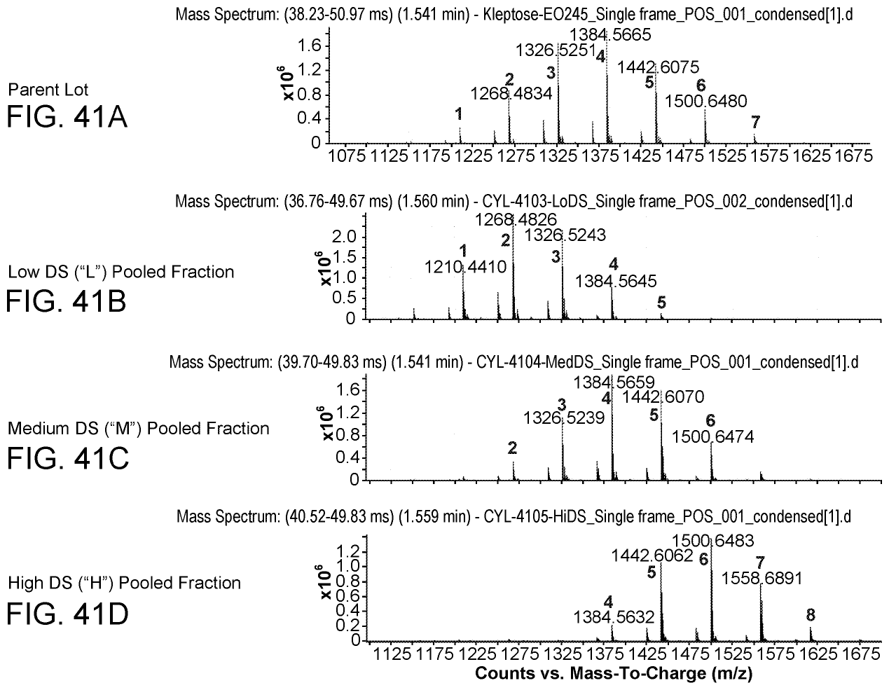

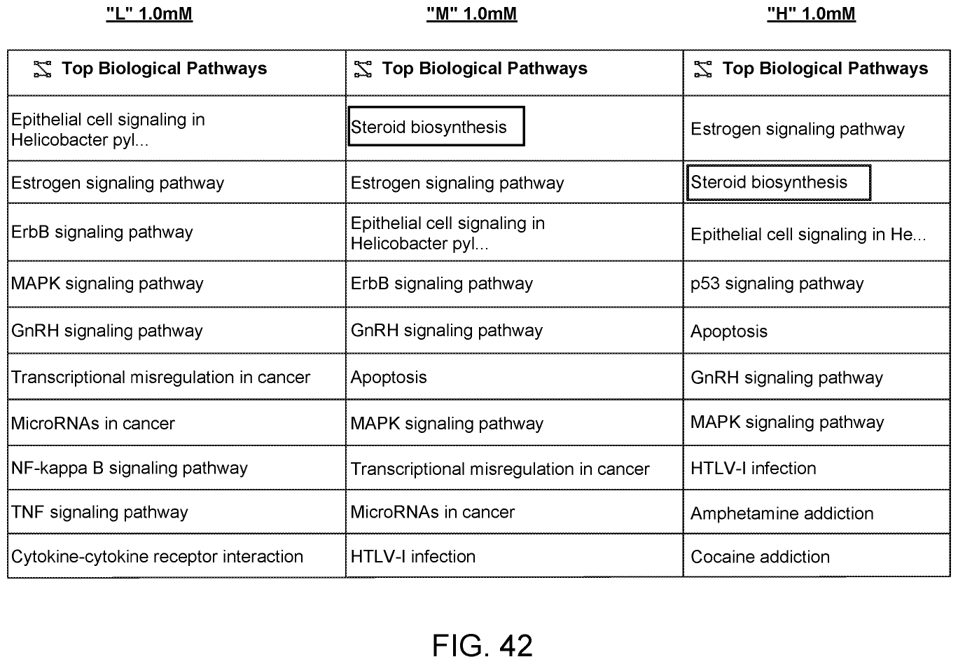

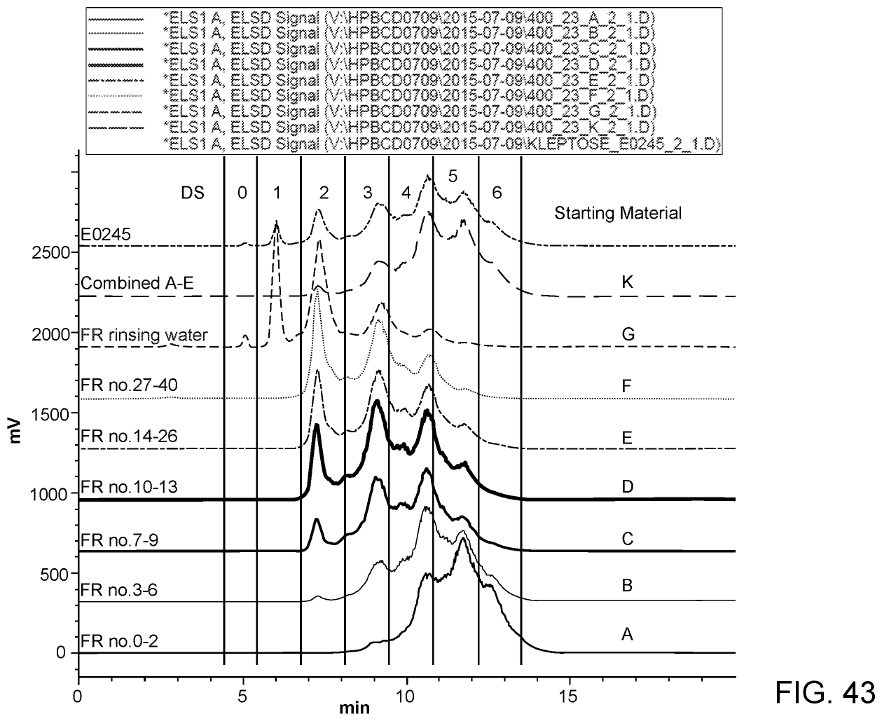

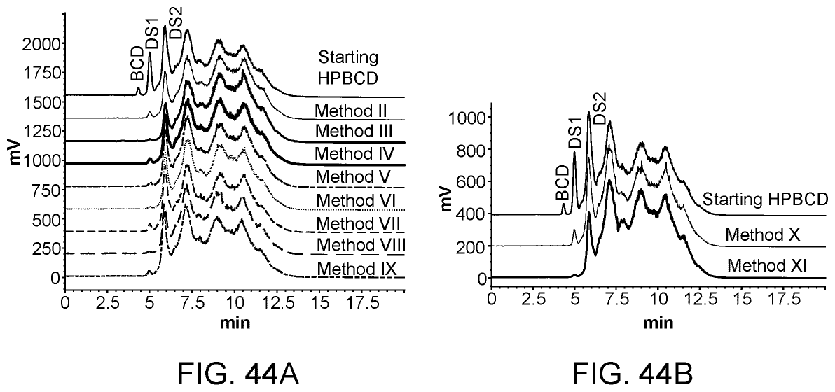

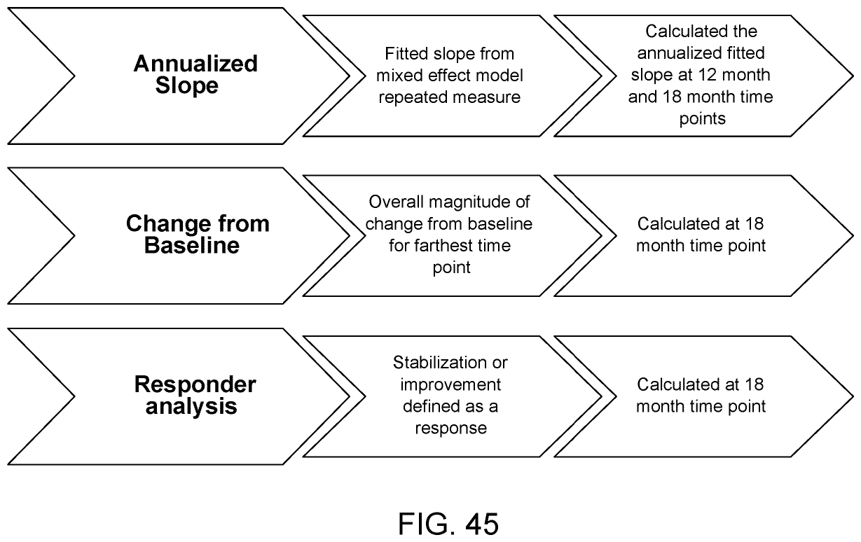

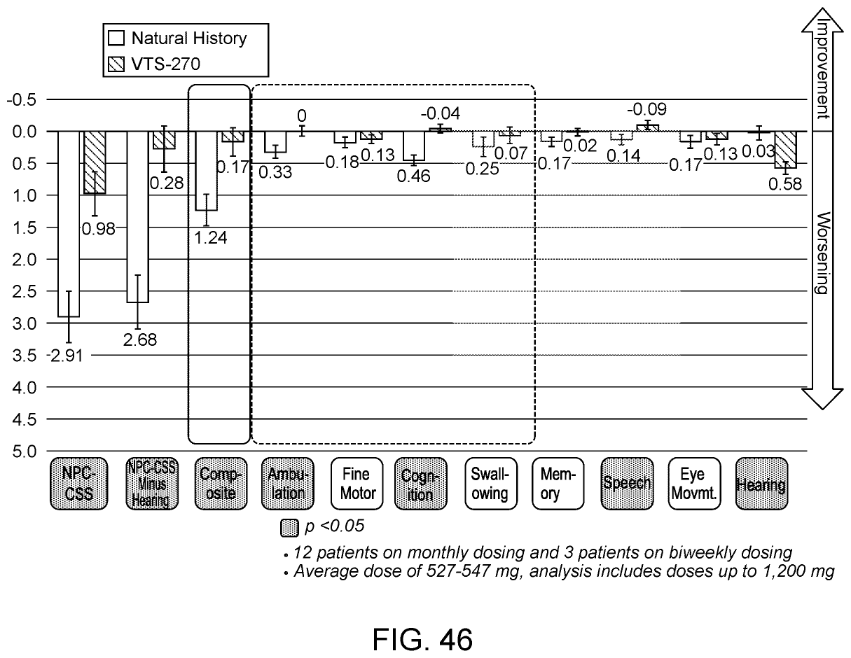

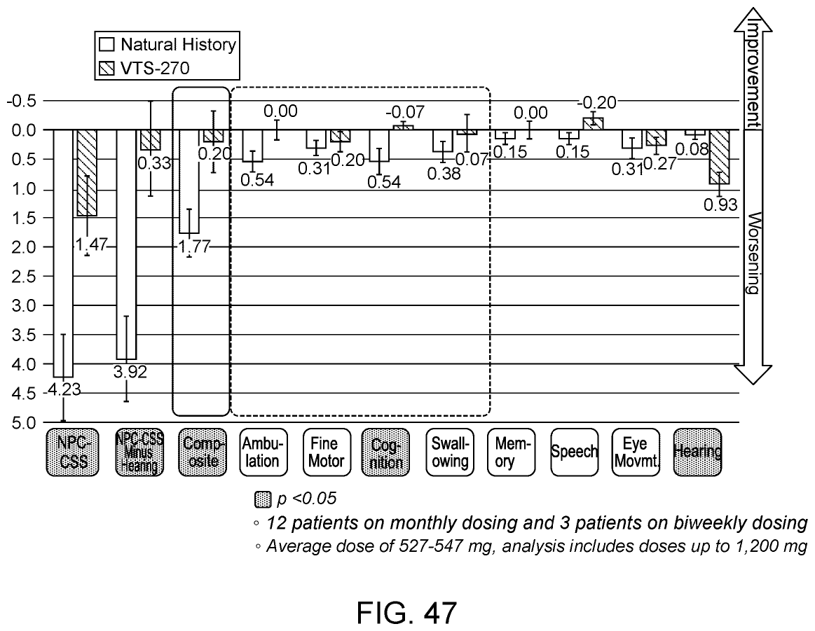

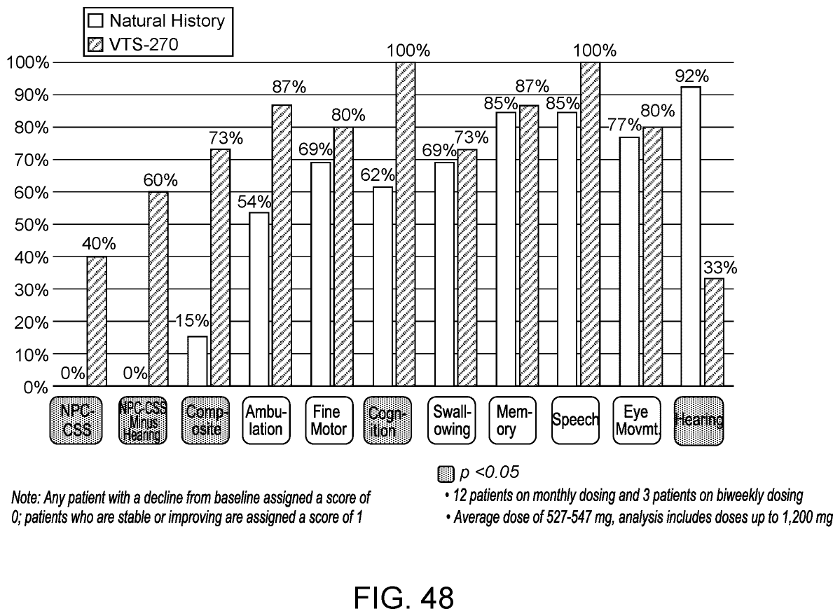

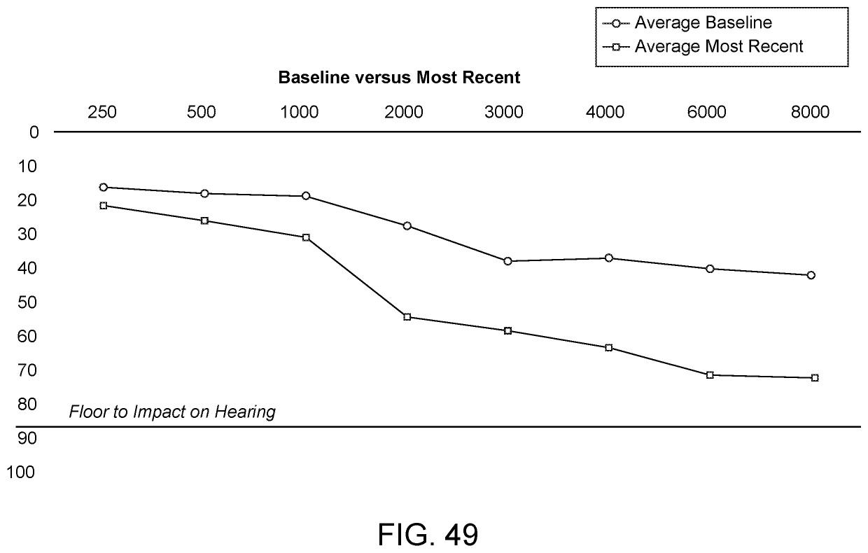

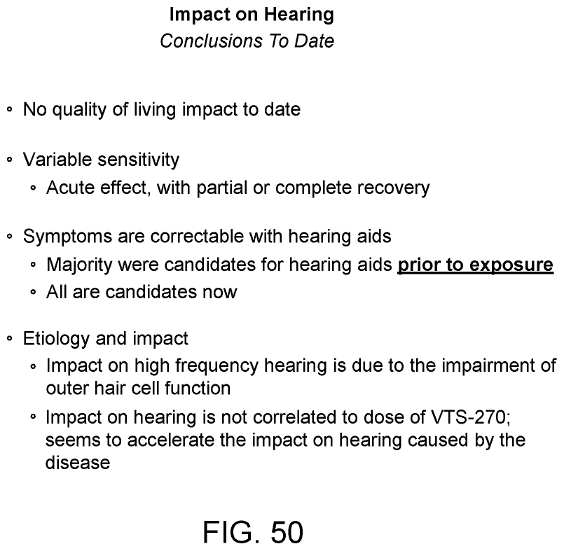

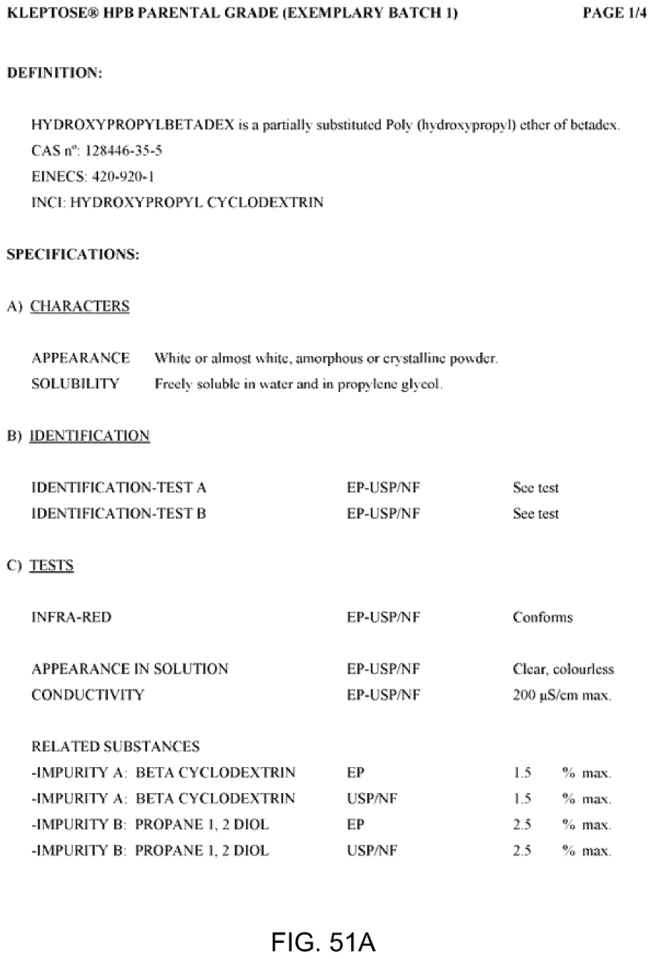

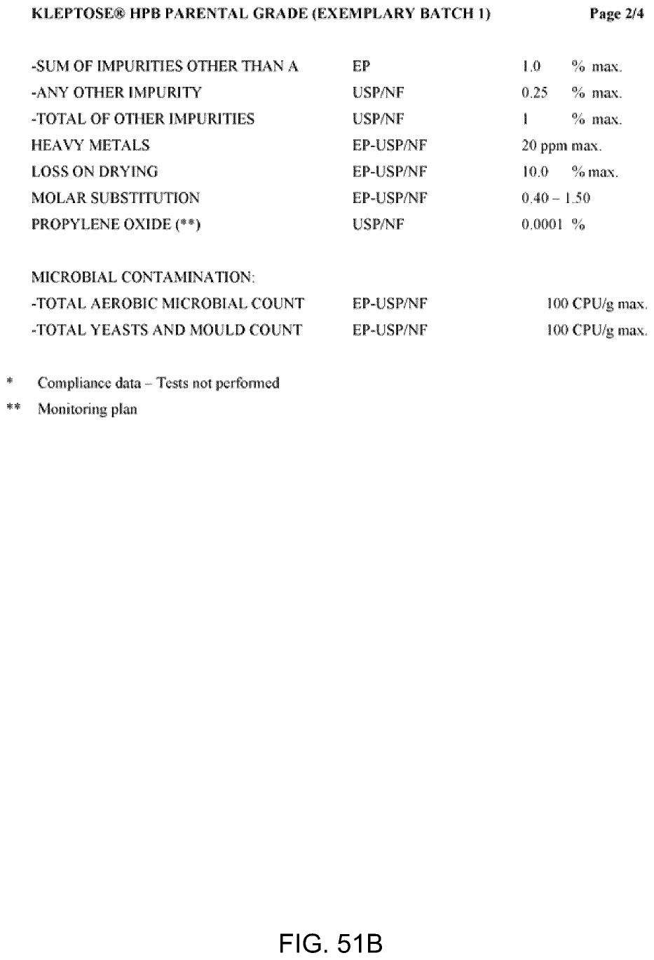

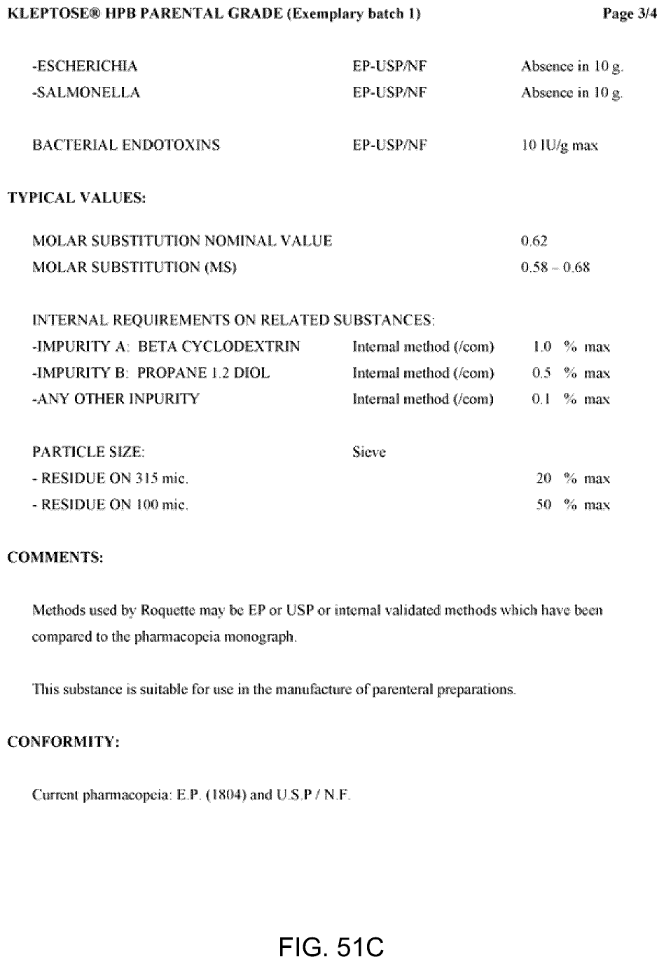

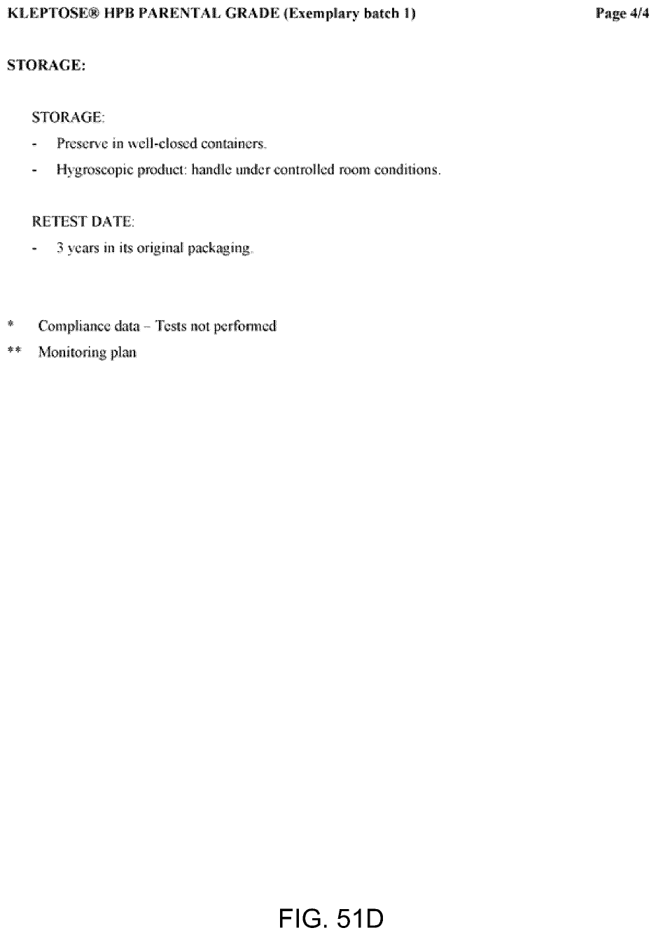

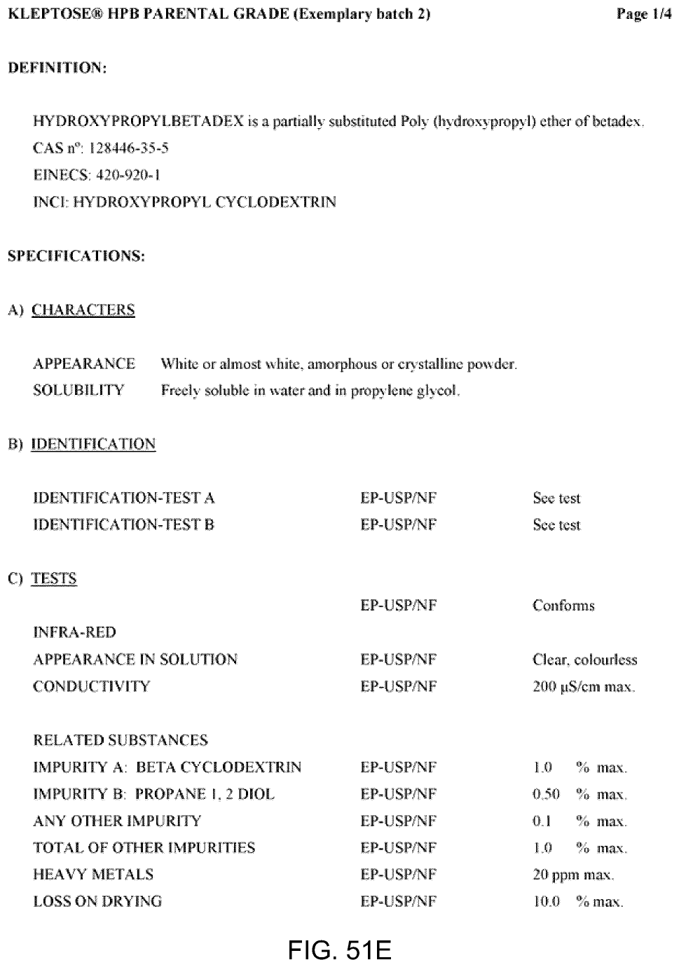

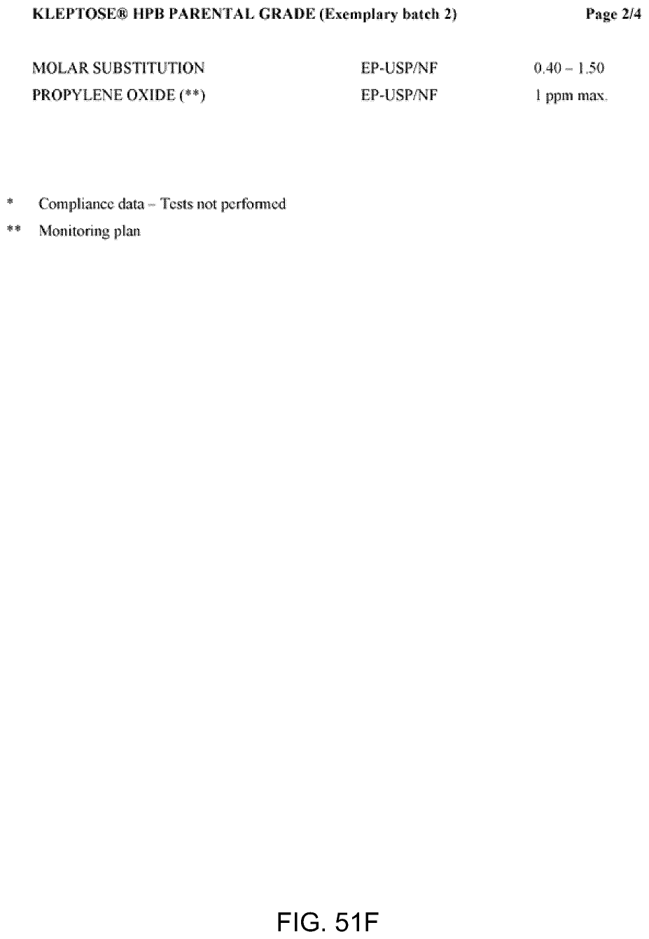

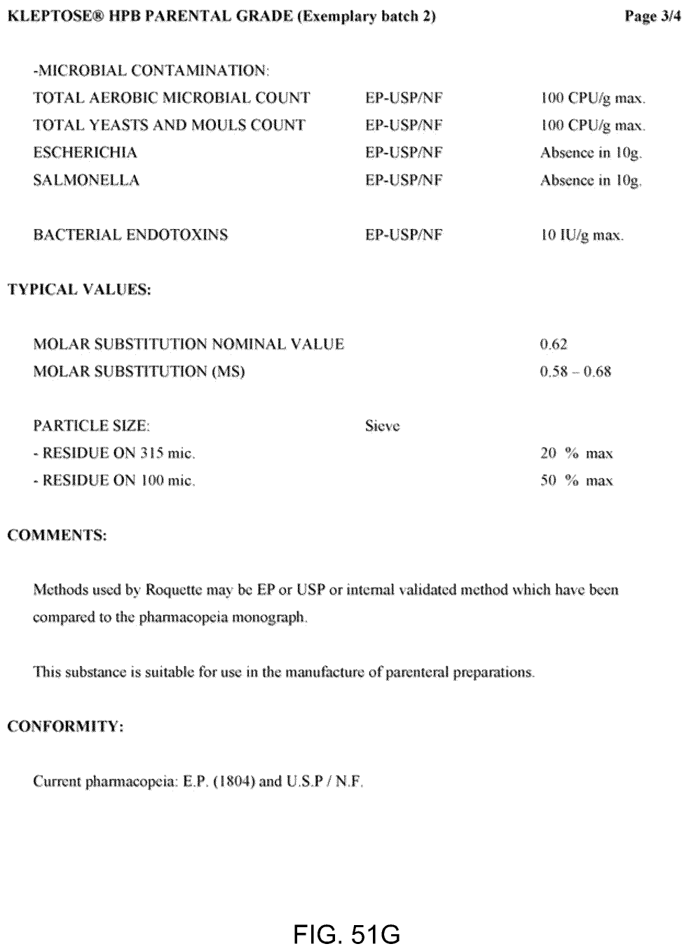

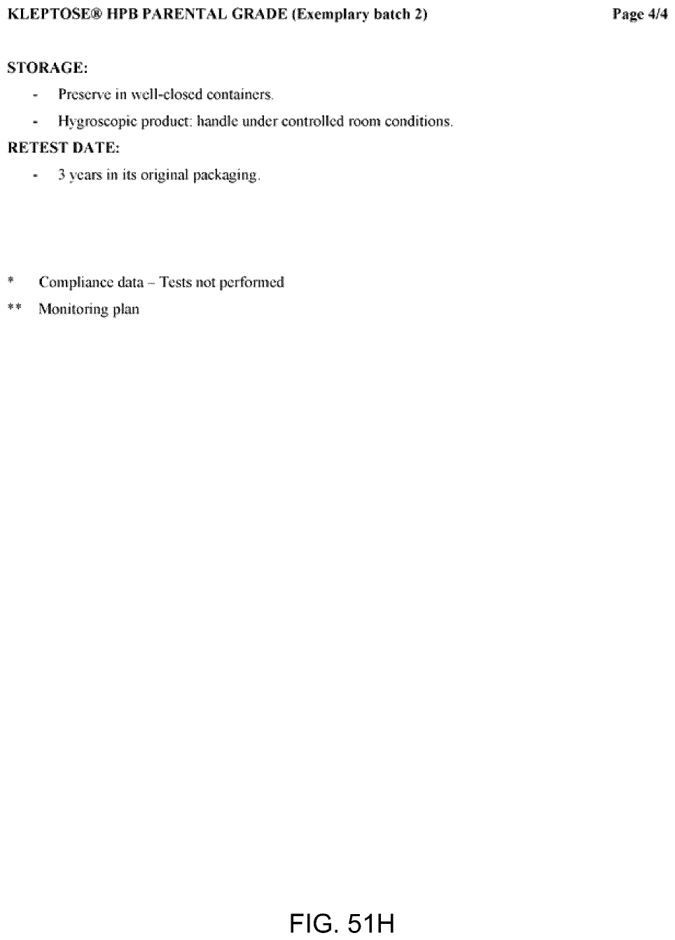

Molecular docking was used to simulate the inclusion mode of MAA and HPβCD. The initial structure of HPβCD was taken from the Cambridge Crystal Database, and the structure of MAA was constructed by GaussianView. The two structures were optimized until there was no imaginary frequency by using the semi-empirical PM3 method without any restrictive factor, and the optimized structure was used as the input configuration for molecular docking. With AutoDock4.2 program, conformational search was carried out for 50 times using the Lamarckian evolutionary algorithm to dock MAA into the cavity of HPβCD. Since the lower the binding energy, the greater the affinity, and the more stable its conformation, after analyzing the conformation search results, the inclusion mode with the lowest binding energy was obtained, as shown in FIG. 10.

FIG. 10 shows that the D ring of MAA enters the cavity from the small opening end of HPβCD, and the hydroxyl on the MAA molecule forms a hydrogen bond (1.676 Å) with the oxygen atom on HPβCD, and finally the A, B, and C rings of MAA penetrate into the cavity of HPβCD. This result is consistent with the results shown by H-NMR.

The above results show that the present invention successfully realizes the inclusion of maackiain (MAA) and hydroxypropyl-β-cyclodextrin (HPβCD), and by simulating gastric juice environment and intestinal juice environment, the results all show that after the inclusion compound was formed, the stability is significantly improved, and the solubility of MAA increases approximately 10 times after inclusion.

### Example 2

8.5 mg of maackiain was accurately weighed, added to a 25 mL round bottom flask to mix with 2 mL of ethanol, stirred at room temperature for 3 h, until the sample was completely dissolved. A certain amount of water was added until the solution in the round-bottomed flask just became turbid, then 43.8 mg of HPβCD was added and the mixture was stirred in darkness for 1 h. The system was filtered to remove unreacted MAA, and the filtrate was then filtered with a 0.45 μm microporous filter membrane, rotarily evaporated to dryness at 60° C., and dried under vacuum to obtain an MAA/HPβCD inclusion compound.

The obtained inclusion compound was tested according to the method of Example 1, and the obtained results are similar to those in Example 1.

### Example 3

8.5 mg of maackiain was accurately weighed, added to a 25 mL round bottom flask to mix with 2 mL of ethanol, stirred at room temperature for 1.5 h, until the sample was completely dissolved. A certain amount of water was added until the solution in the round-bottomed flask just became turbid, then 43.8 mg of HPβCD was added and the mixture was stirred in darkness for 5 h. The system was filtered to remove unreacted MAA, and the filtrate was then filtered with a 0.45 μm microporous filter membrane, rotarily evaporated to dryness at 60° C., and dried under vacuum to obtain an MAA/HPβCD inclusion compound.

The obtained inclusion compound was tested according to the method of Example 1, and the obtained results are similar to those in Example 1.

It can be seen from the above examples that the method provided by the invention successfully realizes maackiain and hydroxypropyl-β-cyclodextrin. The obtained inclusion compound has significantly improved stability and solubility, so that it has a broad application prospect.

The above is only the preferred embodiments of the present invention. It should be pointed out that for those of ordinary skill in the art, numerous improvements and modifications can be made without departing from the principles of the present invention, and these improvements and modifications should also be regarded as the scope of protection of the present invention.

What is claimed is:

1. A method of treating Niemann Pick in a human patient in need thereof comprising administering a composition comprising a mixture of beta-cyclodextrin molecules substituted at one or more hydroxyl positions by hydroxypropyl groups, wherein: the mixture comprises unsubstituted beta-cyclodextrin (“DS-0”), beta-cyclodextrin substituted with one hydroxypropyl group (“DS-1”), and less than 10.0% beta-cyclodextrin substituted with seven hydroxypropyl groups (“DS-7”).

2. The method of claim 1, wherein the mixture comprises less than 2.0% DS-0.

3. The method of claim 1, wherein the mixture comprises less than 1.5% DS-0.

4. The method of claim 1, wherein the mixture comprises less than 4.0% DS-1.

5. The method of claim 1, wherein the mixture comprises less than 3.0% DS-1.

6. The method of claim 1, wherein the composition comprises less than 6 International Units (IU) of endotoxins per gram.

7. The method of claim 1, wherein the composition comprises less than 4 International Units (IU) of endotoxins per gram.

8. The method of claim 1, wherein the composition comprises less than 2 International Units (IU) of endotoxins per gram.

9. The method of claim 1, wherein the composition comprises less than 1 International Units (IU) of endotoxins per gram.

10. The method of claim 1, wherein the mixture has an average molar substitution (“MS”) in the range of 0.40-0.80.

11. The method of claim 1, wherein the mixture has an average degree of substitution (“DSₐ”) of about 3 to about 7.

12. The method of claim 1, wherein the mixture has an average degree of substitution (“DSₐ”) of about 6 to about 7.

13. The method of claim 1, wherein the composition comprises no more than 1 ppm propylene oxide.

14. The method of claim 1, wherein the total amount of other unspecified impurities is less than or equal to 0.05%.

15. The method of claim 1, wherein the composition has a concentration of about 10 mg/mL to about 200 mg/mL.

16. The method of claim 1, wherein the patient is a pediatric patient.

17. The method of claim 1, wherein the patient is an adult patient.

18. The method of claim 1, wherein the composition further comprises a pharmaceutically acceptable diluent.

19. The method of claim 1, wherein the mixture comprises less than 9% DS-7.

20. The method of claim 1, wherein the mixture comprises less than 8% DS-7.

21. The method of claim 1, wherein the mixture comprises less than 7% DS-7.

22. A method of treating Niemann Pick in a human patient in need thereof comprising administering a composition comprising a mixture of beta-cyclodextrin molecules substituted at one or more hydroxyl positions by hydroxypropyl groups, wherein: the mixture comprises unsubstituted beta-cyclodextrin (“DS-0”), beta-cyclodextrin substituted with one hydroxypropyl group (“DS-1”), and from 1% to 10% beta-cyclodextrin substituted with seven hydroxypropyl groups (“DS-7”).

23. The method of claim 22, wherein the mixture comprises less than 2.0% DS-0.

24. The method of claim 22, wherein the mixture comprises less than 1.5% DS-0.

25. The method of claim 22, wherein the mixture comprises less than 4.0% DS-1.

26. The method of claim 22, wherein the mixture comprises less than 3.0% DS-1.

27. The method of claim 22, wherein the composition comprises less than 6 International Units (IU) of endotoxins per gram.

28. The method of claim 22, wherein the composition comprises less than 4 International Units (IU) of endotoxins per gram.

29. The method of claim 22, wherein the composition comprises less than 2 International Units (IU) of endotoxins per gram.

30. The method of claim 22, wherein the composition comprises less than 1 International Units (IU) of endotoxins per gram.
